(19) United States
(12) Patent Application Publication
(10) Pub. No.: US 2020/0187910 A1
(43) Pub. Date: Jun. 18, 2020

(54) ADAPTIVE MULTIFOCUS BEAMFORMING ULTRASOUND METHODS AND SYSTEMS FOR IMPROVED PENETRATION AND TARGET SENSITIVITY AT HIGH FRAME-RATES

(71) Applicant: THE UNIVERSITY OF NORTH CAROLINA AT CHAPEL HiILL, Chapel Hill, NC (US)

(72) Inventors: Gianmarco Francesco PINTON, Chapel Hill, NC (US); Paul Alexander DAYTON, Carrboro, NC (US); David Antonio ESPINDOLA ROJAS, Chapel Hill, NC (US); Fanglue LIN, Chapel Hill, NC (US)

(73) Assignee: The University of North Carolina at Chapel Hill, Chapel Hill, NC (US)

(21) Appl. No.: 16/608,406

(22) PCT Filed: May 9, 2018
(86) PCT No.: PCT/US2018/031837

§ 371 Date: Oct. 25, 2019

Related U.S. Application Data
(60) Provisional application No. 62/503,880, filed on May 9, 2017.

(51) Int. Cl. A61B 8/08; A61B 8/00; A61B 8/06

(52) U.S. Cl. CPC A61B 8/5207; A61B 8/481; A61B 8/469; A61B 8/0808; A61B 8/488; A61B 8/085; A61B 8/4488; A61B 8/06; A61B 8/0891; A61B 8/0825

(57) ABSTRACT

Disclosed is a method and related systems comprising detecting a position of each of two or more targets in a volume to be imaged; generating and directing a single beam of ultrasound energy toward the volume by simultaneously focusing the single beam on each of the two or more the target positions; detecting the ultrasound energy from each of the two or more the target positions; and using the detected ultrasound energy to generate an image of the volume to be imaged. Such as generating a blood flow profile in a blood vessel.

### CROSS-REFERENCE TO RELATED APPLICATIONS

This application claims benefit of U.S. Provisional Patent Application Ser. No. 62/503,880, filed May 9, 2017, herein incorporated by reference in its entirety.

### TECHNICAL FIELD

The presently disclosed subject matter relates generally to methods and systems for generating ultrasound images. In some embodiments, the presently disclosed subject matter relates to methods and systems employing adaptive multifocus beamforming for generating ultrasound images.

### BACKGROUND

In ultrasound imaging the shape of the transmitted sound beam has a large impact on the image quality and frame-rate. Currently, plane wave transmissions, which are very wide, are used to illuminate the whole field of view at a high frame-rate. An advantage of this approach is that the frame-rates can be very high. However, the disadvantage of using such a broad transmit beam is that the image quality is poor and the penetration is low. A focused transmission, which is also currently widely used, sends out a beam that is focused to a single location. It has a higher image quality in terms of contrast and penetration but a lower frame-rate because multiple focused emissions must be used to obtain a single ultrasound image. For plane-wave imaging, on the other hand, only a single emission is necessary to generate an image.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method for generating an ultrasound image of a volume. In some embodiments, the method comprises detecting a position of each of two or more targets in the volume to be imaged; generating and directing a single beam of ultrasound energy toward the volume by simultaneously focusing the single beam on each of the two or more the target positions; detecting the ultrasound energy from each of the two or more the target positions; and using the detected ultrasound energy to generate an image of the volume to be imaged.

In some embodiments, the each of the two or more targets comprises a contrast agent. In some embodiments, the contrast agent comprises one or more microbubble, one or more microdroplet, or a combination thereof. In some embodiments, the detecting a position of each of two or more targets comprises generating and directing a plane wave into the volume. In some embodiments, the detecting a position of each of two or more targets is carried out continually. In some embodiments, the method comprises transmitting pulses according to a sequence, optionally a temporal sequence, which simultaneously focuses the single beam on each of the two or more the target positions. In some embodiments, the method comprises employing an ultrasound transducer comprising a programmable array configured to transmit pulses according to a sequence, optionally a temporal sequence, that simultaneously focuses the single beam on each of the two or more the target positions. In some embodiments, the method comprises repeating the steps at a frame rate of at least about 5,000 frames per second.

In some embodiments, the volume to be imaged comprises a tissue in a subject, optionally wherein the tissue is selected from the group consisting of brain, breast and thyroid tissue. In some embodiments, the volume to be imaged comprises a tumor in a subject, optionally a microvasculature of a tumor, in a subject.

In some embodiments, the presently disclosed subject matter provides an imaging system. In some embodiments, the imaging system comprises: at least one ultrasound transducer configured to detect a position of each of two or more targets in the volume to be imaged, to generate and direct a single beam of ultrasound energy toward the volume by simultaneously focusing the single beam on each of the two or more the target positions, and to detect the ultrasound energy from each of the two or more the target positions; and a processor programmed to analyze data acquired by the ultrasound transducer from the volume in order to output an image from the volume. In some embodiments, the at least one ultrasound transducer comprises two or more ultrasound transducers, optionally wherein each ultrasound transducer is configured to detect a position of each of two or more targets in the volume to be imaged, to generate and direct a single beam of ultrasound energy toward the volume by simultaneously focusing the single beam on each of the two or more the target positions, and to detect the ultrasound energy from each of the two or more the target positions.

In some embodiments, the system comprises a contrast agent, wherein contrast agent is adapted for administration to the volume, such that the each of the two or more targets can comprise a contrast agent. In some embodiments, the contrast agent comprises one or more microbubble, one or more microdroplet, or a combination thereof.

In some embodiments, the at least one ultrasound transducer is configured to detect a position of each of two or more targets by generating and directing a plane wave into the volume. In some embodiments, the at least one ultrasound transducer is configured to detect a position of each of two or more targets continually. In some embodiments, the at least one ultrasound transducer is configured to transmit pulses according to a sequence, optionally a temporal sequence, that simultaneously focuses the single beam on each of the two or more the target positions. In some embodiments, the at least one ultrasound transducer comprises a programmable array configured to transmit pulses according to a sequence, optionally a temporal sequence, which simultaneously focuses the single beam on each of the two or more the target positions. In some embodiments, the system is configured to repeat functions at a frame rate of at least about 5,000 frames per second.

In some embodiments, the system is configured to image a volume comprising a tissue in a subject, optionally wherein the tissue is selected from the group consisting of brain, breast and thyroid tissue. In some embodiment, the system is configured to image a volume comprising a tumor in a subject, optionally a microvasculature of a tumor, in a subject.

In some embodiments, the presently disclosed subject matter provides a non-transitory computer readable medium having stored thereon executable instructions that when executed by the processor of a computer control the computer to perform steps comprising: detecting a position of each of two or more targets in the volume to be imaged; generating and directing a single beam of ultrasound energy toward the volume by simultaneously focusing the single beam on each of the two or more the target positions; detecting the ultrasound energy from each of the two or more the target positions; and using the detected ultrasound energy to generate an image of the volume to be imaged.

In accordance with some embodiments of the presently disclosed subject matter, a method for generating a blood flow profile in a blood vessel is disclosed. In some embodiments, the method comprises: detecting a position and/or motion of a target in the blood flow in a blood vessel to be imaged in a first ultrasound image and in a second ultrasound image, wherein the first ultrasound image and the second ultrasound image are generated by: (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image; and generating a blood flow profile in the blood vessel based on the position and/or of the target in the first frame and the second frame.

In some embodiments, the target comprises a contrast agent. In some embodiments, the contrast agent comprises one or microbubble, one or more microdroplet, or a combination thereof.

In some embodiments, the target comprises two or more targets wherein the generating and directing a beam of ultrasound energy toward the blood vessel and focusing the beam on each of the two or more the targets comprises: generating and directing a single beam of ultrasound energy toward the blood vessel by simultaneously focusing the single beam on each of the two or more the targets. In some embodiments, the method comprises transmitting pulses according to a sequence, optionally a temporal sequence, which simultaneously focuses the single beam on each of the two or more the targets. In some embodiments, the method comprises employing an ultrasound transducer comprising a programmable array configured to transmit pulses according to a sequence, optionally a temporal sequence, that simultaneously focuses the single beam on each of the two or more the targets. In some embodiments, the method comprises repeating the steps (a)-(c) at a frame rate of at least about 5,000 frames per second.

In some embodiments, the blood vessel to imaged comprises a blood vessel in a tissue in a subject, optionally wherein the tissue is selected from the group consisting of brain, breast and thyroid tissue. In some embodiments, the blood vessel to be imaged comprises a blood vessel of a tumor in a subject. In some embodiments, the blood vessel is present in the subject at a depth of at least about 3 centimeters

In some embodiments, generating a blood flow profile comprises determining a velocity of the blood flow, determining a blood vessel pattern, generating a tortuosity measurement for the blood vessel. In some embodiments, generating a blood flow profile in the blood vessel based on the position and/or of the target in the first frame and the second frame comprises applying a pattern matching function to the ultrasound energy from the target in the first image and the second image.

In some embodiments, the blood vessel is in the brain of a subject, and the method comprises applying a correction that refocuses the ultrasound beam as it propagates through a skull of the subject and/or accounts for one or more variation in skull morphology. In some embodiments, the correction is applied twice, first when the beam propagates through the skull after being directed toward the blood vessel, and then when the beam propagates from each of the two or more the target positions for detection.

In some embodiments, the method comprises detecting the position and/or motion of a target in the blood flow in a blood vessel to be imaged in a reference ultrasound image or images and in a subsequent ultrasound image or images, wherein the reference image or images and the subsequent ultrasound image or images are generated by (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image; and generating a blood flow profile in the blood vessel based on the position of the target in the reference image or images and subsequent image or images.

In accordance with some embodiments of the presently disclosed subject matter, an imaging system is disclosed. In some embodiments, the imaging system comprises at least one ultrasound transducer configured to detect a position and/or motion of a target in the blood flow in a blood vessel to be imaged in a first ultrasound image and in a second ultrasound image, wherein the first ultrasound image and the second ultrasound image are generated by: (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image; and a processor programmed to analyze data acquired by the ultrasound transducer to generate a blood flow profile in the blood vessel based on the position and/or of the target in the first frame and the second frame.

In some embodiments, the system comprises a contrast agent, wherein the contrast agent is adapted for administration to the blood vessel. In some embodiments, the contrast agent comprises one or microbubble, one or more microdroplet, or a combination thereof.

In some embodiments, the target comprises two or more targets and wherein the at least one ultrasound transducer is configured to generate and direct a single beam of ultrasound energy toward the blood vessel by simultaneously focusing the single beam on each of the two or more the targets. In some embodiments, the at least one ultrasound transducer is configured to transmit pulses according to a sequence, optionally a temporal sequence, which simultaneously focuses the single beam on each of the two or more the targets. In some embodiments, the at least one ultrasound transducer comprises a programmable array configured to transmit pulses according to a sequence, optionally a temporal sequence, that simultaneously focuses the single beam on each of the two or more the targets. In some embodiments, the at least one ultrasound transducer is configured to repeat the steps (a)-(c) at a frame rate of at least about 5,000 frames per second.

In some embodiments, the system is configured to image a blood vessel in a tissue in a subject, optionally wherein the tissue is selected from the group consisting of brain, breast and thyroid tissue. In some embodiments, the system is configured to image a blood vessel of a tumor in a subject. In some embodiments, the blood vessel is present in the subject at a depth of at least about 3 centimeters In some embodiments, the processor is configured to generate a blood flow profile comprising one or more of a velocity of the blood flow, a blood vessel pattern, and a tortuosity measurement for the blood vessel. In some embodiments, the processor is configured to apply a pattern matching function to the ultrasound energy from the target in the first image and the second image.

In some embodiments, the blood vessel is in the brain of a subject, and the at least one ultrasound transducer is configured to apply a correction that refocuses the ultrasound beam as it propagates through a skull of the subject and/or accounts for one or more variation in skull morphology. In some embodiments, the correction is applied twice, first when the beam propagates through the skull after being directed toward the blood vessel, and then when the beam propagates from each of the two or more the target positions for detection.

In some embodiments, the at least one ultrasound transducer configured to detect the position and/or motion of a target in the blood flow in a blood vessel to be imaged in a reference ultrasound image or images and in a subsequent ultrasound image or images, wherein the reference image or images and the subsequent ultrasound image or images are generated by (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image.

In accordance with some embodiments of the presently disclosed subject matter, a non-transitory computer readable medium having stored thereon executable instruction is disclosed. In some embodiments, the non-transitory computer readable medium having stored thereon executable instructions that when executed by the processor of a computer control the computer to perform steps comprising: detecting a position and/or motion of a target in the blood flow in a blood vessel to be imaged in a first ultrasound image and in a second ultrasound image, wherein the first ultrasound image and the second ultrasound image are generated by: (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image; and generating a blood flow profile in the blood vessel based on the position and/or motion of the target in the first frame and the second frame.

It is thus an object of the presently disclosed subject matter to provide methods and systems employing adaptive multifocus beamforming for generating ultrasound images and methods and systems for generating blood flow profiles.

An object of the presently disclosed subject matter having been stated hereinabove, and which is achieved in whole or in part by the presently disclosed subject matter, other objects will become evident as the description proceeds when taken in connection with the accompanying Figures and Examples as best described herein below.

### BRIEF DESCRIPTION OF THE FIGURES

FIG. 1 is a schematic drawing showing a representative ultrasound system that can employ adaptive multifocus beamforming for generating ultrasound images in accordance with some embodiments of the presently disclosed subject matter.

FIG. 2 is a schematic drawing showing adaptive multifocus beamforming for generating ultrasound images in accordance with some embodiments of the presently disclosed subject matter.

FIG. 3 is a digital image showing an example of super-resolution contrast US in the rat brain, showing resolution of vessels on the order of 10 μm at depths greater than 1 cm. Reproduced from Ericco et al, Nature, 2015.

FIGS. 4A and 4B show CESR imaging in vitro. (FIG. 4A) Acquired beamformed B mode image of 50 micron tube with contrast. (FIG. 4B) Reconstructed CESR image after filtering and centroid detection illustrating substantial resolution enhancement (same scale).

FIG. 5 shows a maximum intensity projection through three-dimensional CESR images of a rat subcutaneous fibrosarcoma (FSA) tumor volume, showing microvascular structure. Analysis of vessel branching (not shown here) demonstrates resolution of separable 25 micron vessels.

FIG. 6 provides a demonstration of a representative embodiment of an adaptive beamforming multi-focus approach in accordance with the presently disclosed subject matter, involving a phantom constructed from 150 μm microtubes in a 6 cm deep ex-vivo tissue phantom. The triple panel to the left of FIG. 6 shows a comparison of multifocus adaptive beamforming versus plane wave and flat focused beam. Left triple panel: (FIG. 6, Panel a) B-mode image from conventional plane wave imaging; (FIG. 6, Panel b) B-mode image from 16 broad focused beams with flat focal depth at 43 mm. (FIG. 6, Panel c) B-mode image from one multifocus beam with two targeting foci at the locations of the microtubes (perpendicular to image plane). Squares and arrows show locations of microtubes at 36 and 53 mm deep. Other squares show tissue location for CTR measurements. The triple panels to the right and top of FIG. 6 show reconstructed images (from ˜35-55 mm only, SVD filtered image. Right triple panel of FIG. 6, top, Panels a2, b2, c2, respectively: SVD filtered images of the tubes from the data presented in FIG. 6, Panel a-FIG. 6, Panel c. Right triple panel bottom of FIG. 6, resultant contrast enhanced super resolution (CESR) images, and zoom-in of super-resolution images of deeper tube. Note that plane wave imaging does not provide sufficient sensitivity at depth (FIG. 6, Panel a3) whereas the multi-focus technique (FIG. 6, Panel c3) achieves sensitivity as good as flat focusing (FIG. 6, Panel b3), and can detect and resolve the 150 micron tube even at 5+ cm with high CTR. (See data in Table 2). Yet, the multi-focus technique achieves a ˜16 fold greater frame rate.

FIG. 7 is a schematic system diagram for 1024 channel imaging system showing integration of four 256 channel subsystems and controllers.

FIG. 8A is a reference B-mode image illustrating the transcranial imaging and showing the skull between a depth of 12-25 mm (top panel), reverberation artefact between 25 and 70 mm (middle panel), and the 150 micron tube which is oriented into the imaging plane (bottom panel)(should be a point) but appears laterally blurred at a 78 mm depth.

FIG. 8B is a graph showing the cross section of the super-resolution images as measured by bubble count for the three imaging configurations, which shows the increased sensitivity of the proposed phase corrected method, which also accurately measures the microtube size.

FIG. 9 shows average difference between consecutives B-mode frames and shows that the proposed phase corrected transcranial super-resolution method resolves a 150 μm diameter tube at 78 mm depth through pig skull at 2.5 MHz. The bubble detection (FIG. 9, Panels a1 through a3) and super-resolution images (FIG. 9, Panels b1 through b3) of the 150 micron microtube imaged transcranially at a depth of 78 mm. The top triple panels, Panels a1 through a3, show the subtraction images used to detect bubble motion zoomed in at the tube location for the three imaging modes: plane wave, Panel a1; conventional focused wave, Panel a2; skull corrected focused wave, Panel a3. The bottom triple panels, Panels b1 through b3, show the super-resolution images and zooms in even further to show fine details. The corrected focused imaging sequence (Panel b3) clearly resolves the microtube with accurate size and position, even through the skull bone and at 78 mm of depth. This ability for precision resolution microvessel imaging through a skull bone is unprecedented in ultrasound imaging and is due to the combined approaches for aberration correction and super resolution imaging.

FIG. 10 shows an exemplary 3-D contrast enhanced super resolution image of rat sarcoma tumor microvasculature, resolving vessels on the order of 20 microns (shown as a maximum intensity projection). Adapted from Lin et al., Theranostics, vol. 7, pp. 196-204, 2016; FOV˜12×30 mm. 4.5 MHz, 220 kPa, 8000 images/slice.

FIG. 11 is a series of panels showing imaging of a 200 micron inner diameter microtube. The 200 micron inner diameter microtube at 57.7 mm depth with a controlled microbubble flow was imaged using in accordance with the presently disclosed subject matter (Panel A). A zoom in of the microtube region is depicted in Panel B. The microbubbles were localized and their positions were tracked to estimate the velocity inside the tube (Panel C). The disclosed TCESR blood velocity technique can estimate the parabolic velocity profiles inside the tube (Panel D). The plot lines correspond to 3 different imposed flows, 5 μL/min; 10 μL/min; and 20 μL/min. The velocity measurements match simplified theoretical predictions based on conservation of mass (Panel E).

FIG. 12 is a series of schematic panels showing coil configuration for continuous arterial spin labeling (CASL) (left), and schematic presentation of the method to measure cerebral blood flow (CBF). High resolution quantitative blood flow maps are obtaining by calculating difference between control and blood tagging signal contrast. Figure extracted from Ajna Borogovac and Iris AsMani, “Arterial Spin Labeling (ASL) fMRI: Advantages, Theoretical Constrains and Experimental Challenges in Neurosciences,” International Journal of Biomedical Imaging, vol. 2012, Article ID 818456, 13 pages, 2012.

### DETAILED DESCRIPTION

The present subject matter will now be described more fully hereinafter with reference to the accompanying Figures, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Reference will now be made in detail to the description of the present subject matter, one or more examples of which are shown in the Figures. Each example is provided to explain the subject matter and not as a limitation. In fact, features illustrated or described as part of one embodiment can be used in another embodiment to yield still a further embodiment. It is intended that the present subject matter cover such modifications and variations. Wherever possible, the same reference numbers will be used throughout the Figures to refer to the same or like parts. The scaling of the Figures does not represent precise dimensions of the various elements illustrated therein.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

While the following terms are believed to be well understood by one of ordinary skill in the art, the following definitions are set forth to facilitate explanation of the presently claimed subject matter.

In some embodiments, the subject imaging according to the presently disclosed subject matter is a human subject, although it is to be understood that the methods described herein are effective with respect to all mammals. More particularly, provided herein is the imaging of mammals, such as humans, as well as those mammals of importance due to being endangered (such as Siberian tigers), of economical importance (animals raised on farms for consumption or another use (e.g., the production of wool) by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Thus, embodiments of the methods described herein include the imaging of livestock and pets.

Following long-standing patent law convention, the terms “a”, “an”, and “the” refer to “one or more” when used herein, including in the claims.

As used herein, the term “about”, when referring to a value or an amount, for example, relative to another measure, is meant to encompass variations of in some embodiments ±20%, in some embodiments ±10%, in some embodiments ±5%, in some embodiments ±1%, and in some embodiments ±0.1% from the specified value or amount, as such variations are appropriate. The term “about” can be applied to all values set forth herein.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

### I. General Considerations

Ultra-Fast and Super-Resolution Imaging.

Recent developments in ultrasound hardware and software have enabled a substantial leap forward in ultrasound imaging technology. New programmable ultrasound systems can utilize software beamformers, parallel and distributed computing architectures, and large onboard memory to perform ultra-fast imaging, on the order of thousands of frames per second, compared to ultrasound systems still utilized in the clinic which are limited to only slightly past 30 frames per second. One such novel technology is “ultrasound localization microscopy”, also referred to herein as contrast enhanced super-resolution (CESR) imaging [5].

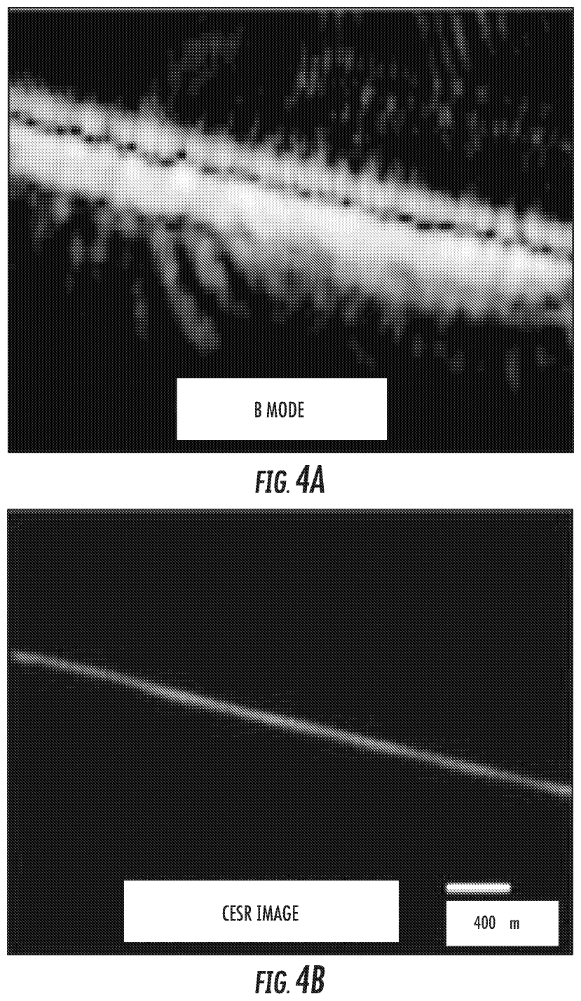

Optical localization microscopy exploits the stochastic blinking of specific fluorescent sources and super-localizes the center of each source by virtue of its separability [3, 4]. By accumulating these center positions over thousands of acquisitions, the resulting image achieves a ten-fold resolution improvement and enables imaging cell membrane and small organelles with a resolution beyond the diffraction limit. In the medical ultrasound domain, different techniques have been investigated to achieve an ultrasound super resolution image, using the blinking of contrast agent microbubbles as an acoustic equivalent of the fluorescent sources. Some groups used highly diluted contrast agents to meet the key requisite of separable microbubble detection [16, 17]. Although these groups obtained super-resolved images using conventional ultrasound scanners, the long acquisition time necessary to perform diluted microbubble super resolution imaging is likely to impinge upon its practicality. The application of ultrafast acquisition and spatiotemporal filtering to separate microbubbles even at a clinically-relevant concentration, by exploiting the decorrelation of microbubbles from a stack of images, has been demonstrated [16, 18]. This technique is a direct analog to fluorescence photoactivation localization microscopy (FPALM) in optics and the acquisition time is more reasonable for clinical translation. By localizing the centers of separable scattering microbubbles, this ultrasound localization microscopy technique allows imaging of microvessels at resolutions as small as ten micrometers, over an order of magnitude smaller than the ultrasound diffraction limit. See FIG. 3, which shows super-resolved vasculature maps of rat brain slices using this super resolution contrast ultrasound technique [5]. This approach has been taken a step further, applying it with a mechanically scanned system to obtain 3-D images of tumor-associated angiogenesis [6]. These results illustrate that the same microvascular abnormalities observed with Acoustic Angiography in tumors can be observed—supporting that super-resolution imaging can be used to image cancer biomarkers in humans. It is of particular note that CESR can be performed effectively at clinical frequencies (data to date has been acquired at 4.5 MHz with a clinical ATL probe), and depths up to 10 cm are theoretically achievable while still retaining resolution better than 100 microns, as long as microbubbles are detectable in original B mode imaging.

Another notable advantage of CESR is that it can be performed at low mechanical indices, less than 0.2, which means that it is a non-destructive imaging technique, and can readily be performed within guidelines for contrast in humans.

Software-Based Ultrasound Systems.

Within the last few years, computational power has finally achieved the performance required to design almost all components of an ultrasound system with dedicated programmable integrated circuits. Consequently, instead of large analog componentry with fixed capabilities, modern ultrasound systems can fit into the case of a simple PC, and still be highly programmable. Commercial vendors such as Verasonics, Cephasonics, and others, are now making programmable ultrasound systems widely available. The high-performance hardware means that these systems can transmit and receive data at rates up to the pulse repetition frequency limit based on speed of sound (thousands of frames per second, depending on tissue depth). This has facilitated super-resolution imaging as described above.

Impact of Ultrasound as a Diagnostic Tool.

While there are clearly clinical applications for MRI, PET, SPECT, and CT that will never be supplanted by acoustics, there are clear clinical applications where ultrasound is well positioned to improve clinical outcome.

For example, breast ultrasound has poor specificity and a high false positive rate, and hence it is not used as a screening tool for breast cancer. Furthermore, ultrasound is challenged to detect breast lesions smaller than a few millimeters. The same can be said about ultrasound's sensitivity to malignant thyroid and prostate cancer.

Improvements in specificity in these fields would have a significant clinical impact. Furthermore, due to the low cost and portability of the next generation of laptop or handheld-sized ultrasound systems, this modality is uniquely poised to expand diagnostic capability to rural and underserved locations and populations worldwide.

Other clinical applications, such as assessing vasa vasorum in atherosclerotic plaques, or assessing angiogenesis in wound healing, may also benefit from high-resolution microvascular ultrasound imaging. Furthermore, the applications of high resolution microvascular imaging in pre-clinical cancer research are readily apparent.

Contrast Ultrasound in the Clinic.

Although several years ago (2007) there had been a concern about the safety of ultrasound contrast due to events in a clinical trial of a new agent, an overwhelming amount of more recent evidence from large clinical studies has shown that contrast ultrasound is very safe; [19-21] in fact, it is much safer than other commonly used techniques, such as coronary angiography, exercise ECG, or myocardial scintigraphy.[22] Over the past decade, the FDA has reversed most of the limitations indicated with contrast enhanced ultrasound based on accumulated safety data from hundreds of thousands of contrast ultrasound exams, and in 2016, the FDA approved contrast ultrasound approval include liver radiological applications in addition to cardiology. Contrast ultrasound is widely gaining renewed enthusiasm, and many sites have ongoing clinical trials for other applications, such as contrast ultrasound for renal applications and breast lesion differentiation.

Angiogenesis as a Biomarker of Malignancy.

More than 40 years ago, Judah Folkman published his seminal paper postulating that tumor angiogenesis was a requirement for tumor growth beyond 2-3 millimeters.[7] Since then, thinking has evolved and it is now understood that angiogenesis is required for the development of even much smaller malignancies in many environments [9, 10, 23-27]. In 2006, Dr. Folkman further elaborated on his initial postulate, suggesting that “tumor cell proliferation alone, in the absence of angiogenesis, can give rise to dormant, microscopic tumors of 1 mm³ or less, but these in situ cancers are harmless to the host” [27]. Hanahan and Weinberg have more recently stated “Historically, angiogenesis was envisioned to be important only when rapidly growing macroscopic tumors had formed, but more recent data indicate that angiogenesis also contributes to the microscopic premalignant phase of neoplastic progression, further cementing its status as an integral hallmark of cancer” [9]. The angiogenic vascular remodeling caused by the presence of cancer cells is characterized by vascular enlargement, increased vessel density, vascular malformations, and increased vessel tortuosity [28]. Most importantly, it occurs very early in tumor development, and thus is an early biomarker for cancer [8-10, 28, 29].

Several key observations related to cancer-associated vascular abnormalities support working in microvascular imaging. Furthermore, we have validated each of these observations, previously based largely on optical microscopy data, with ultrasound microvascular imaging [1]. Researchers have observed that drastic changes in surrounding microvessel morphology initiate early in tumor development, when tumors are very small, using optical microscopy [9, 10, 23-27]. The significance of this effect is that presence of abnormal vessels is an early indicator of cancer presence, even for micro-tumors. Prior studies using Acoustic Angiography imaging in spontaneous rodent models of breast cancer have confirmed this observation—significant differences in microvascular morphology can be visualized even 2-3 micron tumors, compared to healthy tissue [1, 2, 13, 14].

The tortuous morphologies of vessels are not limited to the boundaries of the tumor and are observed well outside lesions boundaries [10, 28]. This has been illustrated in animal models and as well as in humans, and in both large and microscopic tumors by previous investigators [10, 27]. This observation has been validated using ultrasound microvascular imaging as well [14]. The significance is that the “angiogenic” fingerprint presents a larger imaging target than just the tumor mass, thereby potentially improving our detection/differentiation ability.

Vessel abnormalities are observed with many types of cancer, in different organs, and across species [1, 10, 30]. The significance of this observation is that a method to detect cancer based on vascular abnormalities would be applicable to a very wide range of pre-clinical and clinical studies.

Changes in microvascularity preceed changes in tumor size in response to therapy, providing an early biomarker of response to therapy [31, 32]. This phenomena has been observed with ultrasound imaging of microvessels also, showing the ability to detect response in “responder” tumors earlier than indicated with measurements of tumor volume alone [33]. The clear advantages of CESR include superior resolution at clinically relevant depths (3-8 cm), the ability to use low mechanical index (<0.2) as well as standard commercial transducers, can be combined with high frame rate 3-D imaging.

Ultrasound-Localization Microscopy/Contrast Enhanced Super Resolution (CESR) Imaging.

The methods described herein employ the imaging techniques of Ultrasound Localization Microscopy, also called Contrast Enhanced Super-Resolution Imaging (CESR), including 3-D CESR implementation. Thus, in some embodiments, the term “super-resolved” refers to the use of CESR. Fast 3-D CESR is employed with a matrix transducer and is combined with unique approaches such as multi-focus adaptive beamforming to increase sensitivity.

High-Frame Rate Plane Wave Imaging.

CESR employs high frame rate imaging, in the range of hundreds to thousands of frames per second (fps). This employs interrogation of the imaged medium with a large number of transmit pulses, typically in the form of a plane wave that insonifies the entire image space. Image reconstruction for plane wave transmits employs capturing and storage of the echo returns from each individual element in the transducer for later processing. Verasonics, a commercial source, has developed plane wave reconstruction in its research systems, with capturing of multi-frame receive channel data and high speed software reconstruction techniques. This has enabled the development of a number of new ultrasound techniques, including shear wave elastography, high frame rate Doppler and vector Doppler imaging, flow imaging using speckle tracking, functional brain flow imaging, and more. High speed data acquisition, storage and processing capabilities are provided with Verasonics systems.

Adaptive Multifocus Beamforming for Improved Penetration and Target Sensitivity at High Frame-Rates.

Plane wave transmissions, which are currently used in super resolution imaging, illuminate the whole field of view with a single planar emission. The advantage of this method is that the frame-rates can be very high; however, the disadvantage of using such a broad transmit beam is that the image quality is poor and the penetration is low. In accordance with some embodiments of the presently disclosed subject matter an adaptive multifocus method circumvents these imaging limitations while preserving the same high frame-rates in plane wave imaging. Representative results using this technique (see Examples below) show that by using this method, the imaging quality is significantly improved in terms of contrast-to-tissue ratio (CTR), as one representative measure. This translates the imaging technology from the mouse/rat, which has a shallow imaging depth, to human targets such as the breast and other organs, which require deeper imaging.

Real-Time High Channel Count High-Frame Rate Acquisition.

Three dimensional plane wave imaging can employ matrix arrays with a large number of elements operating very quickly. All received data from the individual elements of the transducer are captured and processed for image reconstruction. A 1024 channel programmable acquisition system capable of acquiring more than 5000 frames/second is provided. This can be employed volume imaging with a 32×32 matrix array at thousands of Hertz. This performance is several times what is currently available commercial devices to coherently capture the full RF data set from large element count arrays. Data is stored in large arrays of fast solid state drives, and consolidated via high speed network transfers for image reconstruction and bubble tracking.

Assessing Vascular Morphologies as a Function of Tumor Progression in Clinically Relevant Models of Disease.

With the development of 3-D high resolution microvascular imaging tools, a diagnostic approach for human diseases characterized by abnormalities of the microvasculature is provided. Imaging tools in mice genetically engineered to mimic the progression of human breast cancer are used, and observations with high-resolution optical microscopy are validated.

Clinical Diagnostic Technique.

The ability to use microvessel angiogenesis imaging as a local indicator of the likelihood of malignant cancer may provide an innovative clinical diagnostic tool. Although ultrasound will likely never be used for whole-body screening, it is already utilized for imaging target areas such as breast, thyroid, testes, ovaries, and prostate. The ability to use microvascular ultrasound imaging as a sensitive method for screening at-risk patients, for guidance of biopsy, or for ultimately the early detection of subresolution micro-tumors would be highly innovative.

### II. Methods, Systems and Computer Readable Media

To date, all super-resolution contrast imaging is performed with plane wave imaging. This technique is fast, but has poor sensitivity as it uses low energy unfocused beams, and thus is challenged to image clinically relevant concentrations of contrast at depth. The presently disclosed subject matter provides in some embodiments methods that combine the advantages of the imaging quality of a focused transmission with the high frame-rates of a plane wave transmission. The presently disclosed subject matter can therefore circumvent the plane-wave imaging limitations while preserving its high frame-rates.

In some embodiments, the presently disclosed subject matter employs a two-step process. First, the target positions in the imaging field are detected with a conventional imaging transmission. Second, a multifocal beam is designed to simultaneously focus a single transmit beam to the target positions. Since this is a single beam this second transmission can be repeated at high frame-rates. By combining the multiple foci into a single transmission the same frame-rate as plane wave imaging can be achieved. However, the imaging quality is significantly improved in terms of contrast and penetration, which improves the target detection of the imaging system. The imaging quality is significantly improved in terms of contrast-to-tissue ratio (CTR), for example. This translates the imaging technology from the mouse/rat, which has a shallow imaging depth, to human targets such as the breast and other organs, which require deeper imaging.

In some embodiments, the presently disclosed subject matter provides a method for generating an ultrasound image of a volume. In some embodiments, the method comprises detecting a position of each of two or more targets in the volume to be imaged; generating and directing a single beam of ultrasound energy toward the volume by simultaneously focusing the single beam on each of the two or more the target positions; detecting the ultrasound energy from each of the two or more the target positions; and using the detected ultrasound energy to generate an image of the volume to be imaged.

In some embodiments of the presently disclosed methods, each target, including each of the two or more targets, comprises a contrast agent. In some embodiments, the contrast agent comprises one or microbubble, one or more microdroplet, or a combination thereof.

In some embodiments of the presently disclosed methods, detecting a position of each of two or more targets comprises generating and directing a plane wave into the volume. In some embodiments, the presently disclosed subject matter detecting a position of each of two or more targets is carried out continually.

In some embodiments, the presently disclosed methods comprise transmitting pulses according to a sequence, optionally a temporal sequence, which simultaneously focuses the single beam on each of the two or more the target positions. In some embodiments, the presently disclosed methods comprise employing an ultrasound transducer comprising a programmable array configured to transmit pulses according to a sequence, optionally a temporal sequence, that simultaneously focuses the single beam on each of the two or more the target positions. In some embodiments, the presently disclosed methods comprise repeating the steps at a frame rate of at least about 5,000 frames per second.

In some embodiments, the blood vessel to be imaged comprises a blood vessel in a tissue in a subject. In some embodiments, the tissue is selected from the group consisting of brain, breast and thyroid tissue. In some embodiments, the blood vessel to be imaged comprises a blood vessel of a tumor in a subject.

In some embodiments, the presently disclosed methods comprise generating a profile of blood flow in a blood vessel, optionally in a subject. In some embodiments, the methods comprise detecting a position and/or motion of a target in the blood flow in a blood vessel to be imaged in a first ultrasound image and in a second ultrasound image; and generating a blood flow profile in the blood vessel based on the position and/or motion of the target in the first frame and in the second frame. In some embodiments, the first ultrasound image and the second ultrasound image are generated by: (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image.

In some embodiments, additional images are generated and compared, including a series of images (thousands to millions). Thus, in some embodiments, the method comprises detecting the position and/or motion of a target in the blood flow in a blood vessel to be imaged in a reference ultrasound image or images and in a subsequent ultrasound image or images, wherein the reference image or images and the subsequent ultrasound image or images are generated by (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image; and generating a blood flow profile in the blood vessel based on the position of the target in the reference image or images and subsequent image or images. Thus, in some embodiments, the presently disclosed subject matter is used to generate images not just of microvessels but the also images of the flow within them.

In some embodiments, generating a profile of blood flow in a blood vessel can comprise determining a velocity of the blood flow; determining a blood vessel pattern; generating a tortuosity measurement for the blood vessel; or any combination of the foregoing. In some embodiments, pattern matching functions are applied that detect position and velocity between ultrasonic acquisitions. By way of example and not limitation, to calculate, a blood flow profile, such as a velocity profile, microbubbles are detected within the vessel. Then, the map of their locations at one instance in time is shifted and correlated to a map of their positions at a later instance in time. For a specific bubble the shift that yields the maximum correlation is used to determine bubble motion. By way of elaboration and not limitation, microbubbles are flowing in a blood vessel and an aspect of the presently disclosed subject matter is to detect the microbubbles. Once the bubbles are detected at one instance in time, then their location in a second instance in time can be detected. Then, the distance and the time are used to obtain the velocity. In some embodiments, this is done by taking two acquisitions, two images, and detecting one or more microbubbles within each image. Once one or more microbubbles are detected the signals of the one or more microbubbles can be correlated between the two images. This provides a position. In some embodiments, the correlation involves comparing the similarities from a region of the first image to a region of the second image and moving the images around until a good fit is obtained.

In some embodiments of the presently disclosed methods, the blood vessel is in the brain of a subject, and the method comprises applying a apply a correction that (1) refocuses the ultrasound beam as it propagates through a skull of the subject and/or (2) accounts for one or more variation in skull morphology. In some embodiments, the correction is applied twice, first when the beam propagates through the skull after being directed toward the blood vessel, and then when the beam propagates from each of the two or more the target positions for detection. In some embodiments, an acoustic simulation tool is used to determine the acoustic path of sound between the transducer emission and the target. A computed tomography (CT) data set of an individual skull is converted into a map of the acoustical properties of the skull. This acoustic map, in conjunction with the simulation tool, is used to determine the appropriate delay profiles across the transducer face that will generate a focused emission experimentally. This correction profile can be determined by comparing the profile distorted by the skull to a spherical profile using, for example, a) correlation-based pattern matching algorithms or b) by detecting the phase crossing above a certain threshold, for example, a normalized amplitude. This correction profile is specific to an individual skull morphology. In some embodiments, a filter that reduces off axis clutter is applied to the simulated data.

In some embodiments, the blood vessel is present in the subject at a depth of at least about 3 centimeters (cm), including 3 to 6 cm.

Blood flow profiles can be used in any manner as would be apparent to one of ordinary skill in the art upon a review of the instant disclosure. By way of example and not limitation, vascular networks around tumors can be assessed. Neurological stimulation, such as stimulation of regions of the brain can be assessed. Thus, in some aspects, the presently disclosed subject matter provides for functional imaging of a volume, including a volume comprising a blood vessel, including a blood vessel in a subject.

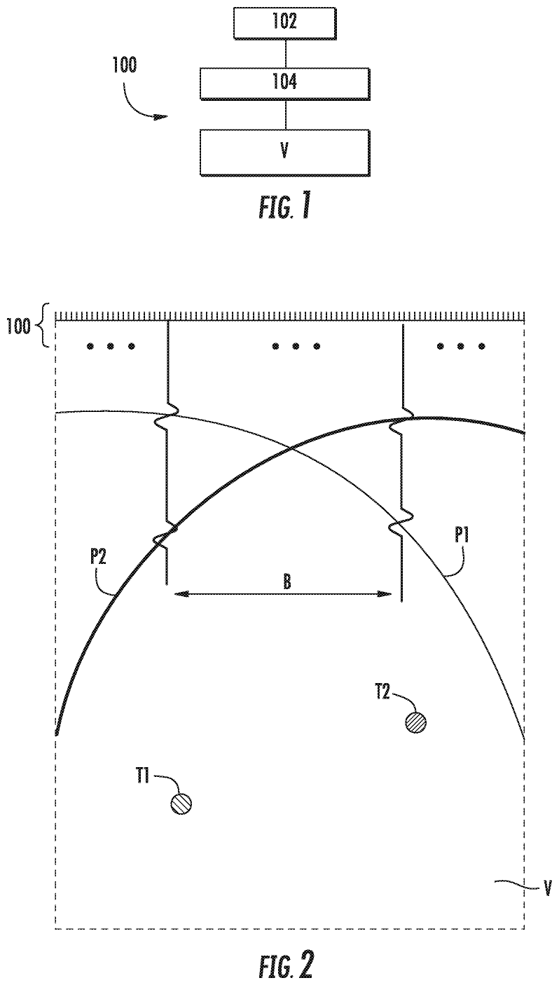

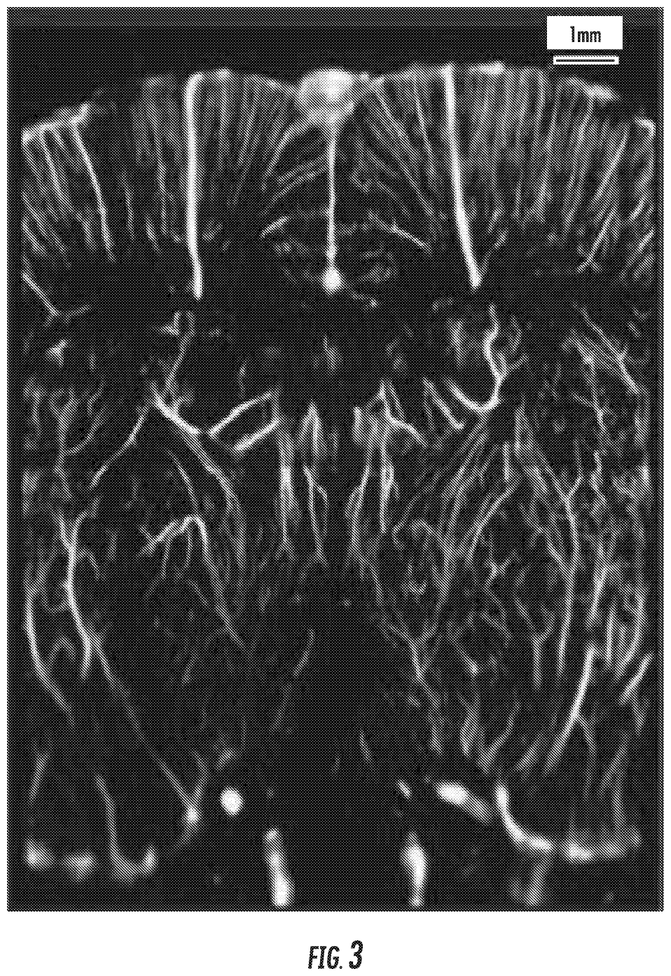

Referring to FIGS. 1 and 2, a system 100 for generating an ultrasound image of a volume V and/or a blood vessel is shown. System 100 is used to detect a position of each of two or more targets, such as targets T1 and T2 shown in FIG. 2, in the volume V to be imaged. System 100 can comprise at least one ultrasound transducer 104 configured to detect a position of each of two or more targets T1 and T2 in the volume V to be imaged, to generate and direct a single beam B of ultrasound energy toward the volume V by employing simultaneous multifocal profiles P1 and P2 designed to simultaneously or concurrently focus the single beam B on each of the two or more the target positions T1 and T2, and to detect the ultrasound energy from each of the two or more the target positions T1 and T2. System 100 comprises a processor 102 programmed to analyze data acquired by the ultrasound transducer 104 from the volume V in order to output an image from the volume V. In some embodiments, system 100 comprises two or more ultrasound transducers 104.

In some embodiments, system 100 comprises a contrast agent, wherein contrast agent is adapted for administration to the volume V, such that the each of the two or more targets T1 and T2 can comprise a contrast agent. In some embodiments, system 100 can comprise microbubbles, microdroplets, or a combination thereof, wherein the microbubbles, microdroplets, or a combination thereof are adapted for administration to the volume V, such that each of the two or more targets T1 and T2 can comprise microbubbles, microdroplets, or a combination thereof. In some embodiments, the contrast agent can comprise microbubbles, microdroplets, or a combination thereof.

Continuing with reference to FIGS. 1 and 2, in some embodiments, the at least one ultrasound transducer 104 is configured to detect a position of each of two or more targets T1 and T2 by generating and directing a plane wave into the volume V. In some embodiments, the at least one ultrasound transducer 104 is configured to detect a position of each of two or more targets T1 and T2 continually. In some embodiments, the at least one ultrasound transducer 104 is configured to transmit pulses according to a sequence, optionally a temporal sequence, that simultaneously focuses the single beam B on each of the two or more the target positions T1 and T2. In some embodiments, the sequence comprises multifocal profiles P1 and P2. In some embodiments, the at least one ultrasound transducer 104 comprises a programmable array configured to transmit pulses according to a sequence, optionally a temporal sequence, that simultaneously focuses the single beam B on each of the two or more the target positions T1 and T2. In some embodiments, the sequence comprises multifocal profiles P1 and P2. In some embodiments, system 100 is configured to repeat functions at in a manner that provides a super-resolved image. In some embodiments, system 100 is configured to repeat functions at a frame rate that provides a super-resolved image. For example, in some embodiments, system 100 is configured to repeat functions at a frame rate of at least about 5,000 frames per second.

In some embodiments, system 100 is configured to image a volume V comprising a tissue in a subject. In some embodiments, system 100 is configured to image a volume V comprising a blood vessel in a subject. In some embodiments, the tissue is selected from the group comprising brain, breast and thyroid tissue. In some embodiments, system 100 is configured to image a volume V comprising a tumor in a subject, optionally a microvasculature of a tumor, in a subject. In some embodiments, the tumor is in the brain of a subject.

In some embodiments, system 100 is configured to generate a profile of blood flow in a blood vessel, optionally in a subject. In some embodiments, system 100 can comprise at least one ultrasound transducer 104 configured to detect a position of a target in the blood flow in a blood vessel to be imaged in a first ultrasound image and in a second ultrasound image; and to generate a blood flow profile in the blood vessel based on the position of the target in the first frame and in the second frame. In some embodiments, the first ultrasound image and the second ultrasound image are generated by: (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image. In some embodiments, additional images are generated and compared.

In some embodiments, system 100 can comprise a processor 102 configured to generate a profile of blood flow in a blood vessel comprising a velocity of the blood flow; determining a blood vessel pattern; generating a tortuosity measurement for the blood vessel; or any combination of the foregoing. In some embodiments, processor 102 is configured to apply pattern matching functions to super-resolved bubble signals that detect position and velocity between ultrasonic acquisitions. By way of example and not limitation, to calculate the velocity profiles, microbubbles are detected within the vessel. Then, the map of their locations at one instance in time is shifted and correlated to a map of their positions at a later instance in time. For a specific bubble the shift that yields the maximum correlation is used to determine bubble motion. By way of elaboration and not limitation, microbubbles are flowing in a blood vessel and an aspect of the presently disclosed subject matter is to detect the microbubbles. Once the bubbles are detected at one instance in time, then their location in a second instance in time can be detected. Then, the distance and the time are used to obtain the velocity. In some embodiments, this is done by taking two acquisitions, two images, and detecting one or more microbubbles within each image. Once one more microbubbles are detected the signals of the one or more microbubbles can be correlated between the two images. This provides a position. In some embodiments, the correlation involves comparing the similarities from a region of the first image to a region of the second image and moving the images around until a good fit is obtained.

In some embodiments of the presently disclosed methods, the blood vessel is in the brain of a subject, and system 100 comprises at least one ultrasound transducer 104 configured to apply a correction that (1) refocuses the ultrasound beam as it propagates through a skull of the subject and/or (2) accounts for one or more variation in skull morphology. In some embodiments, the correction is applied twice, first when the beam propagates through the skull after being directed toward the blood vessel, and then when the beam propagates from each of the two or more the target positions for detection. In some embodiments, an acoustic simulation tool is used to determine the acoustic path of sound between the transducer emission and the target. A computed tomography (CT) data set of an individual skull is converted into a map of the acoustical properties of the skull. This acoustic map, in conjunction with the simulation tool, is used to determine the appropriate delay profiles across the transducer face that will generate a focused emission experimentally. This correction profile can be determined by comparing the profile distorted by the skull to a spherical profile using, for example, a) correlation-based pattern matching algorithms or b) by detecting the phase crossing above a certain threshold, for example, a normalized amplitude. This correction profile is specific to an individual skull morphology. In some embodiments, a filter that reduces off axis clutter is applied to the simulated data.

In some embodiments, the presently disclosed subject matter provides a non-transitory computer readable medium having stored thereon executable instructions that when executed by the processor of a computer control the computer to perform steps comprising: detecting a position of each of two or more targets in the volume to be imaged; generating and directing a single beam of ultrasound energy toward the volume by simultaneously focusing the single beam on each of the two or more the target positions; detecting the ultrasound energy from each of the two or more the target positions; and using the detected ultrasound energy to generate an image of the volume to be imaged.

In some embodiments, the presently disclosed subject matter provides a non-transitory computer readable medium having stored thereon executable instructions that when executed by the processor of a computer control the computer to perform steps comprising: detecting a position of a target in the blood flow in a blood vessel to be imaged in a first ultrasound image and in a second ultrasound image; and generating a blood flow profile in the blood vessel based on the position of the target in the first frame and in the second frame. In some embodiments, the first ultrasound image and the second ultrasound image are generated by: (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image. In some embodiments, additional images are generated and compared.

### III. Examples

The following Examples provide further illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Example 1

### Clinically Relevant Super-Resolution Imaging Methods

This Example relates to improving super-resolution bubble detection and sensitivity at clinical depths, while preserving high frame rates; adapting motion correction algorithms to an imaging approach; obtaining velocity vector information; and evaluating bubble parameters.

Validation of Resolution Advancement Using CESR Imaging.

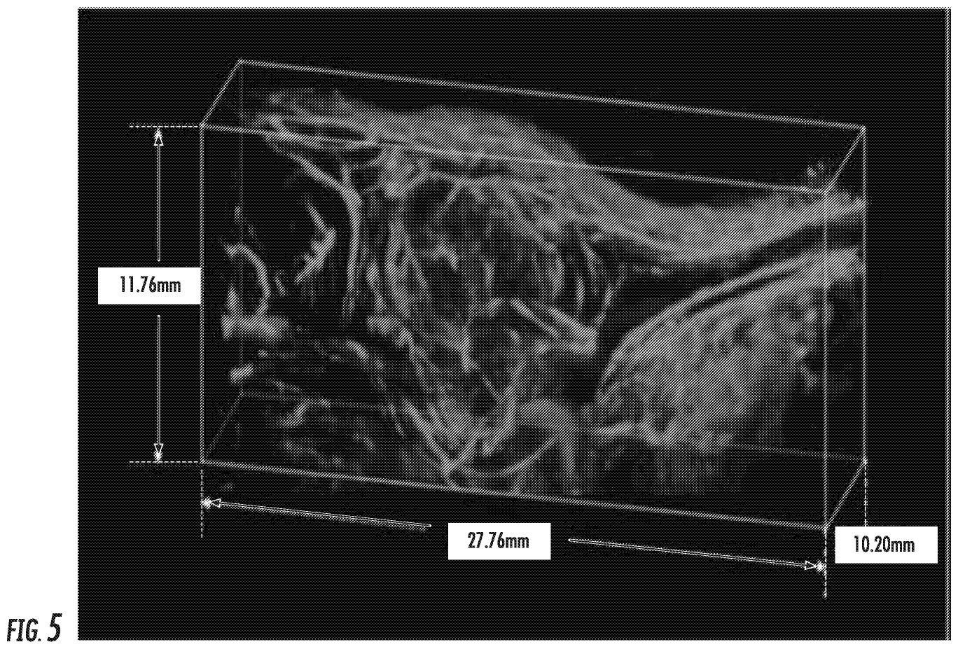

Contrast enhanced super-resolution (CESR) imaging of tumors has been performed using a Verasonics Vantage system (Verasonics Inc., Redmond, Wash., United States of America) with a 128 channel L11˜5 linear probe, using plane-wave imaging at a pulse repetition frequency of 500 Hz. The transmitted pulses were 1 cycle sinusoids at 4.5 MHz with a rarefactional pressure of 220 kPa (mechanical index=0.1). This low mechanical index was chosen to minimize bubble destruction under high frame rate insonification. A high-pass spatiotemporal singular value decomposition (SVD) filter is applied to detect the decorrelation of bubbles, yielding individual sources on the filtered images. This spatiotemporal filter can discriminate bubble signals whose spatial coherence is low from tissue signals whose spatial coherence is high because their temporal variations affect many neighboring pixels the same way.[34] Hysteresis thresholding is used to localize the bubbles on the filtered images. Bubble centers are detected and center positions from all the frames are accumulated to get a super-resolution image, with a pixel size of 10 μm×10 μm, for each scan slice. FIGS. 4a and 4b show before and after processing from a microtube of 50 μm inner diameter. Table 1 illustrates the advancement in resolution of CESR compared to traditional b-mode imaging.

TABLE 1
Comparison of measured tube size
with B mode imaging and
CESR technique-From [6]

| | B mode Imaging | | CESR | |
|---|---|---|---|---|
| Actual | Measured | Error | Measured | Error |
| (μm) | Size (μm) | (%) | Size (μm) | (%) |
| 150 | 171 | 14 | 140 | 6.7 |
| 75 | 171 | 128 | 65 | 14 |
| 50 | 171 | 242 | 40 | 20 |

Preliminary Data—In Vivo 3D CESR Studies.

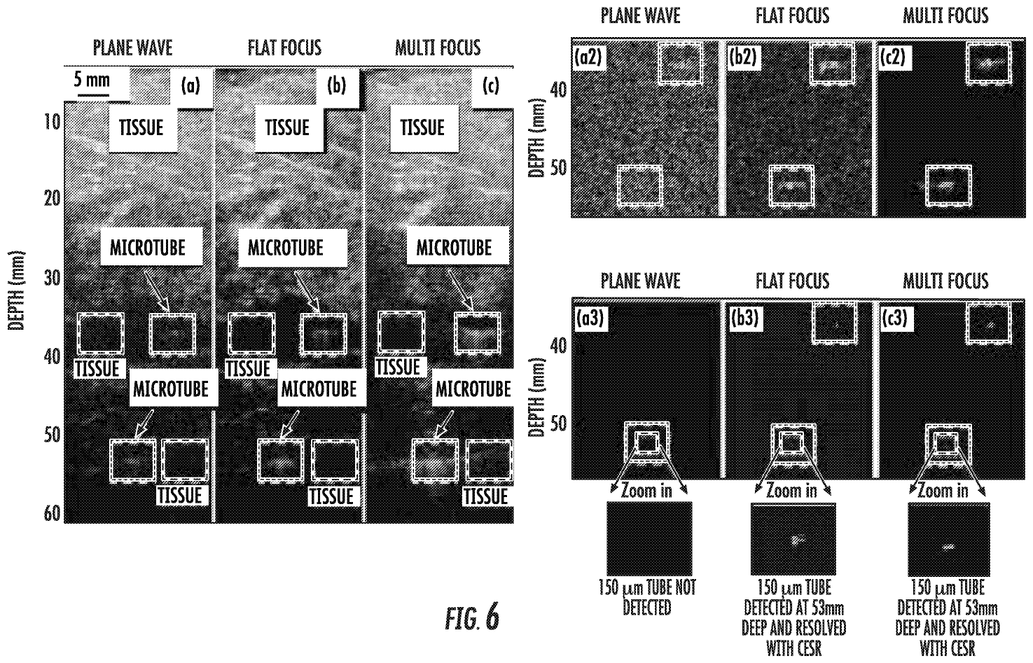

Data are acquired similar to described above for in vitro studies, except the transducer is mounted to a motorized precision motion stage synchronized with the imaging system for 3D scanning. 8,000 images are acquired for each scan slice and slice step size is 200 μm. IQ data are saved for post-processing. CESR images are obtained through offline post-processing on beamformed IQ data. We have been imaging rats with the FSA fibroscarcoma tumor model (as described previously [12]) using this method. Frames with breathing induced motion artifacts are excluded by calculating the frame-to-frame cross-correlation of a chosen region of tissue signals. FIG. 5 shows the maximum intensity projection from 3-D CESR image of a rat fibrosarcoma tumor microvasculature, resolving vessels on the order of 20 microns. CESR imaging of three tumor bearing rats and three control (healthy) rats, followed by vessel segmentation of 379 vessels, showed statistically significant differences in vascular tortuosity between the populations[6], which upholds similar observations using Acoustic Angiography in both rat and mouse models of cancer [1, 2]. Thus, the CESR data confirms that this technique will be useful for assessing angiogenic vascular abnormalities. However, unlike Acoustic Angiography which is limited in penetration depth and resolution (˜150 μm at 2 cm), CESR has the potential to achieve tenfold better resolution at several times the depth, a significant improvement for clinical studies.

### Example 2

### Adaptive Beamforming CESR for Improved Sensitivity and Resolution in Deep Tissues

To date, all super-resolution contrast imaging is performed with plane wave imaging. This technique is fast, but has poor sensitivity as it uses low energy unfocused beams, and thus is challenged to image clinically relevant concentrations of contrast at depth.

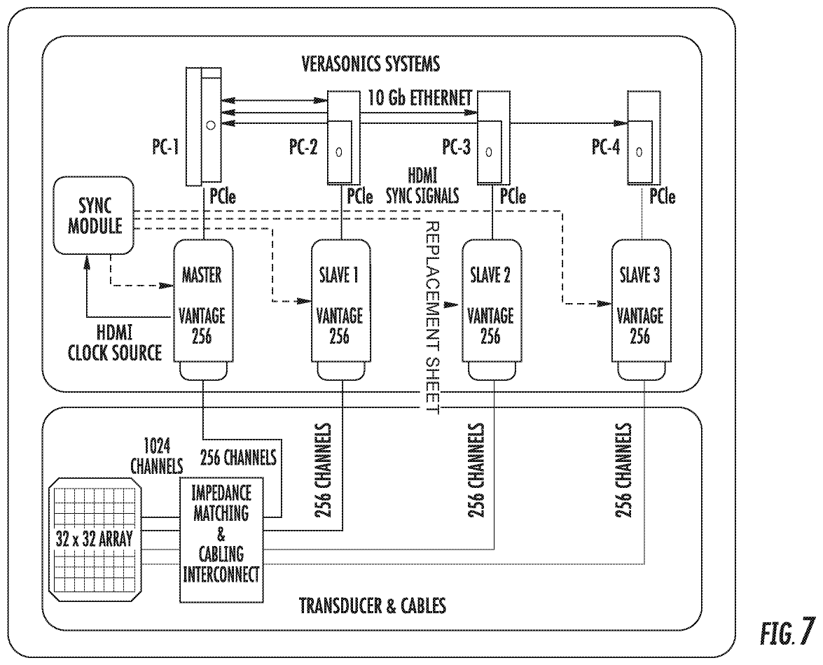

This Example compares an adaptive beamforming technique in accordance with the presently disclosed subject matter with standard plane wave imaging and flat focus imaging. Beef tissue was immersed in degassed water. Two thin-walled microtubes (Paradigm Optics Inc., Washington, United States of America) with inner diameter of 150 μm were embedded in the steak at depths of ˜36 mm and 53 mm. Lipid-shelled microbubbles were pumped through the microtubes, with the aid of an infusion pump (Harvard Apparatus, Holliston, Mass., United States of America) at 50 μL/min. Imaging was performed using a Verasonics Vantage system (Verasonics Inc., Redmond, Wash., United States of America) with a L7-4 linear probe, operating at 50 dB dynamic range. The transmitted pulses were 2 cycle sinusoids at 5.2 MHz. Flat focus imaging used 16 focused beams with a flat focal depth at 43 mm. Adaptive multifocus imaging used one beam with two simultaneous targeting foci at the locations of the two microtubes. The beam repetition rate remains 1428 Hz, leading to a frame rate of 1428 Hz, 89 Hz and 1428 Hz for the three techniques, respectively. Contrast to tissue ratio (CTR) was calculated from the mean signal amplitude of the contrast in the tube compared the mean signal amplitude of the background region at the same depth. Both images and measured contrast to tissue (CTR) values clearly demonstrated that the adaptive multifocus imaging provides equivalent or better contrast to tissue ratio (˜10 dB at 36 mm and ˜8 dB improvement at 53 mm depth) than focused imaging, while keeping the same high frame rate as plane wave imaging (over 16× fold faster frame rate than focused imaging). The result is the ability to perform CESR imaging at significant depth in tissue, without a substantial reduction in frame rate. See FIGS. 6a-6c3.

TABLE 2
Comparison of CTR from Plane waving
imaging, flat focus imaging and
multifocus imaging before reconstruction.
(Fig 6a-6c). Upper and lower
tubes at 36 and 53 mm deep, respectively.

| | Plane wave | Flat focus | Multi focus |
|---|---|---|---|
| Upper microtube | 5.7 dB | 15.3 dB | 15.7 dB |
| Lower microtube | 7.8 dB | 15.1 dB | 15.2 dB |

### Example 3

### Methods: In-Vitro Testing and Analysis

In some embodiments, testing is performed using 256 channel Verasonics systems and using microvessel flow phantoms, with vessel sizes of 200, 150, 50, and 25 microns with precision spacing as measured with an optical microscope in tissue-mimicking phantoms or with muscle tissue (steak and chicken breast) to approximate in-vivo tissue. Microbubbles similar to those available under the trademark DEFINITY™ are prepared according to previously published techniques [1, 2, 35, 36]. Bubble concentrations and distributions are measured by an Accusizer 780 particle sizer, and are pumped through the target phantom with a calibrated flow pump (Harvard Apparatus PhD2000). Acoustic calibrations are performed with a calibrated 200 μm needle hydrophone (HNA-0200, Onda, Inc). Metrics for evaluating performance include image-determined resolution compared to known tube diameters and spacing, as well as CTR, as a function of imaging, bubble parameters, and depth.

### Example 4

### Methods: Adaptive Simultaneous Multifocus Beamforming

The presently disclosed adaptive multifocus methods combine the advantages of the imaging quality of a focused transmission with the high frame-rates of a plane wave transmission, as demonstrated in Example 1 above. Instead of transmitting a plane wave, a single adaptive beam simultaneously targets multiple bubbles with a single emission. In some embodiments, this is achieved via a two-step process. First, the bubble positions in the imaging field or volume are detected with a conventional imaging transmission and with intensity thresholding. Second, a multifocal beam is designed to focus a single transmit beam to multiple bubble positions. Since this is a single beam this second transmission can be repeated at an ultrafast frame-rate to track the bubbles as they move within the vessels. That is, by combining the multiple foci into a single transmission the same frame-rate as plane wave imaging can be achieved. However, the imaging quality is significantly improved in terms of contrast to noise ratio, signal to noise ratio, and CTR, which improves the bubble detection and penetration of the imaging system.

The conventional imaging transmissions to track the bubble positions can be done at regular intervals with high-speed image reconstruction. Lookup tables with multiple focal configurations are generated. The number of bubbles that can be effectively targeted with a single multifocus beam is optimized. For a single target the transmit configuration corresponds to a conventional focus and for an infinite number of targets the transmit configuration corresponds to a plane wave. The configuration of the multifocus transmit is therefore evaluated in terms of optimal target number, bubble concentration, pulse amplitude, transmit rate, and beam profile. This parameter space is investigated experimentally with the Verasonics hardware and in silico with FULLWAVE™, an ultrasound simulation tool developed to model ultrasound propagation and imaging with high accuracy, and which can rapidly prototype the sequence development in 2D and 3D [37, 38].

### Example 5

### Methods: Motion Correction

Since CESR imaging requires thousands of frames acquired per slice, tissue motion can readily corrupt the data. Image registration algorithms based on the cross-correlation of the RF data of successive frames acquired at high frame rate are adaptively evaluated. This method has been shown to be effective for compensating for breathing induced motion during acquisition, as described previously [39, 40]. The incorporation of motion compensation methods preserves the moving frames instead of excluding them as done in preliminary studies. It increases the number of detected bubbles for each slice, and therefore improve resolution and reduce the acquisition time. As an alternative, generation of super resolution images with high temporal resolution by calculating the high order statistics of blinking microbubble signals [41] is explored. This approach may reduce the required acquisition time several fold for each imaging slice. This alternative approach can also mitigate the need for motion compensation.

### Example 6

### Methods: Evaluation of Bubble Parameters

To evaluate parameters for eventual clinical imaging, testing of our approach is performed as a function of bubble concentration. Whether bubble size has an effect is also evaluated. FDA approved contrast agents have different mean diameters. LUMASON™ contrast agents have a mean diameter of 1.5-2.5 microns [42], DEFINITY™ contrast agents have mean diameter from 1-3 microns [43], and OPTISON™ contrast agents have an average diameter listed as 3-4.5 microns [44]. Our studies have previously shown that larger bubbles with a lower resonant frequency and a substantially larger scattering cross section can be more readily detected [36], although these differences have not been assessed in super-resolution imaging. Clinical CESR may benefit from use of larger bubbles to optimize sensitivity, whether these arise from manufacturing differences or intended size tailoring.

Lipid shelled in-house microbubbles are prepared at one, three, and five micron mean diameters and CESR sensitivity is evaluated as a function of bubble size, with constant concentration. Sized microbubble preparations are prepared via centrifugal sorting as previously described [36], and characterized with an ACCUSIZER™ 780 particle sizer.

### Example 7

### Methods: Obtaining Microvascular Flow Information

A bubble velocity vector is utilized to detect microvascular flow [45]. This approach provides flow vectors which can be utilized to provide additional quantitative data regarding microvessel pattern, for example, fluctuations in tumor blood flow which are known to cause hypoxia and re-oxygenation affecting tumor progression and response to treatment [46, 47]. Flow vector mapping can also provide a separate tortuosity measurement for microvessels with opposite slow flow directions. This flow calculation method is based directly on the ability to accurately detect the position of single bubbles within the vessel. Since the bubble position and frame rate is known the velocity vector can be extracted almost directly. Individual bubbles are tagged and velocity vectors are associated within each vessel. This data is aggregated into flow profiles along the length of each vessel. Rejection methods for confounding data, such as when two bubbles paths intersect, are also investigated.

Preliminary bubble localization work used hysteresis thresholding, therefore the choice of the threshold values was empirical. An alternative strategy that would be more accurate is PSF fitting. This approach is also implemented, as described by Desailly et al. [18].

### Example 8

### Hardware for 3-D Super-Resolution Imaging

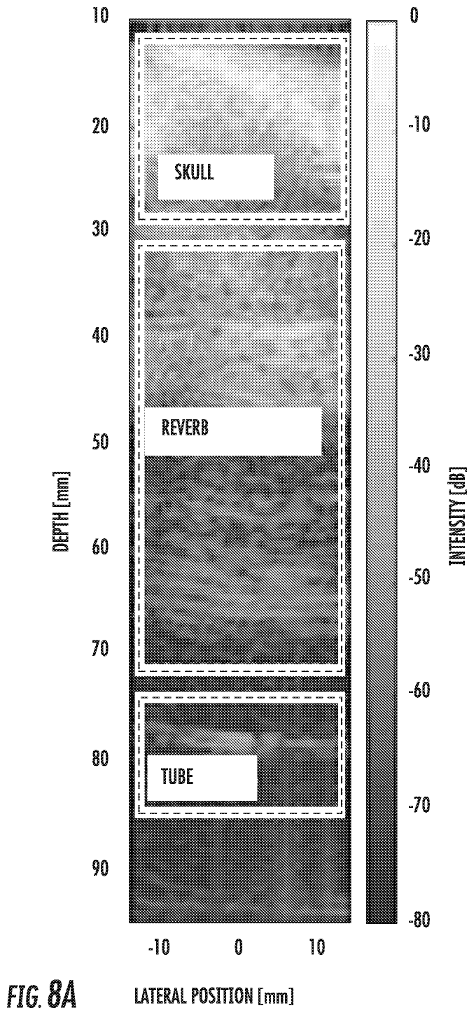

Hardware is provided for ultrafast acquisition of 1024 channel data from a 2-D array, management of the large ultrafast 3-D data stream, implementation and evaluation of super resolution imaging on the ultrasound platform, and generation of 3-D data from a 2-D matrix array at clinically useful frame rates. A 1024 channel system for high-frame rate acquisition and adaptive beamforming is thus provided. CESR is initially developed on a mechanically scanned 1-D array, followed by development on a subset of the 1024 element 3 MHz matrix array (32×32). CESR imaging using the entire matrix array is then implemented, at full 1024 channel volume frame rates of greater than 5000 volumes per second. To accomplish this substantial data processing task, the matrix transducer is interfaced to four 256 channel Verasonics systems, operating synchronously with a single master 250 MHz clock (FIG. 7). This will allow simultaneous coherent RF data acquisitions from all elements of the 1024 element array. Individual element RF data are then digitized, filtered and stored into large memory arrays located in each Verasonics system. Since a single transmit/receive acquisition can generate more than a megabyte of data, thousands of acquisitions per second require storage of gigabytes per second. This high data rate capture is made possible by distributing the storage over the multiple Verasonics systems.

This ultra-high performance programmable 1024 channel ultrasound system comprises four 256 channel Vantage research systems, each with their own high performance computer. One Vantage system acts as a master, while the other three systems act as slaves. The systems are synchronized to the 250 MHz clock of the master through an external sync module that buffers and distributes the master clock and trigger signals to all slaves. The clock synchronization allows for coherent signal sampling across all 1024 receive channels of the combined units. The 1024 elements of the matrix array are grouped into four sets of 256 elements, for cabling to the four Vantage units.

A high performance dual 3.1 GHz Xeon processor based computer with 20 available processing cores and 256 GB of RAM memory controls each Vantage unit. Each Vantage unit also has 3 terabytes of high speed solid state drive storage for rapid storage (up to 6 GB/sec) of acquired RF data. The master unit computer is also equipped with a high end GPU module for additional processing power and an extra 12 terabytes of RAID disk storage for data archiving. For communication and data transfers between units, each unit has a 10 Gb Ethernet module for connection to a local network. Processing of RF and reconstructed image data can be both distributed and centralized. Each of the four custom Vantage units has substantial processing power with their multi-core CPUs. This distributed processing capability is leveraged for real-time image acquisition and data processing. For CESR processing that requires the full data set, the Vantage slave units transfer each unit's data to the master unit's computer for further processing. The master computer can then use the powerful GPU module to facilitate rapid data processing of large data sets. For storing both raw and processed data associated with the CESR method, the master computer can utilize the large 12 TB RAID storage unit.

The control of acquisition and processing is managed by synchronized sequence programs that run concurrently on each Vantage unit. Synchronization is performed by trigger signals that connect the master and slave units through the external sync module. The control software allows operation of the entire system from the master computer console. For shorter acquisition sequences, all unit acquisition and processed data can be saved to local RAM in real-time, allowing for capture of several seconds of data. For longer sequences, software is developed for each unit's rapid transfer of data to the local solid-state drives. This may require somewhat reduced acquisition rates, but will allow for minutes of collection for RF and processed data.

For safety during eventual clinical use all units have electrical power isolation transformers to minimize leakage. For elimination of ground loops, connections between units utilize optical fiber cabling. Hardware and controller PCs are mounted onto a custom rolling cart.

### Example 9

### Methods: Adaptive Multifocus Beamforming in 3-D

Three dimensional image space contains millions of voxels, and to obtain fast reconstruction rates, the image reconstruction processing is distributed amongst the multiple Verasonics systems described in Example 8. Each system can reconstruct 256 channel data at a rate of approximately 10 million voxels per second, generating voxel IQ data that can then be combined to obtain the image from the full 1024 element array. These bubble images will not need the fine resolution of a typical ultrasound image, and the voxel density can be lowered to help meet the processing speed requirements. If necessary, it may also be possible to use only a subset of the 1024 element array to obtain bubble positions, further reducing data processing and transfer requirements.

The flexibility built into the 1024 channel 3D imaging system allows flexibility. If the data from the various Vantage units were required to be consolidated before processing, the rate of data transfer to the master unit could be a bottleneck. Fortunately, the distributed processing capability of the system allows preliminary processing and reduction of the data sets prior to consolidation. The Vantage systems also have the ability to perform bandwidth sampling of band-limited RF signals, resulting in data reductions of 2 to 1 or better. For example, if the 3 MHz 1024 element transducer has a bandwidth of less than 100%, it can be sampled at 6 MSPS instead of the 12 MSPS required for 200% bandwidth. Since each unit has access to a subset of elements representing one fourth of the array, it is also possible to make reference images with lower spatial resolution in each unit. These reference images may be sufficient for bubble tracking and localization, allowing dynamic transmit beamforming to be locally computed. As mentioned earlier, voxel density can be easily controlled, trading off image reconstruction times with spatial resolution. Also, since very large quantities of raw RF data can be stored in real-time, it becomes feasible to collect data with limited real-time processing aimed only at targeting and use offline processing for performing the more complex spatial filtering and flow visualization processing.

### Example 10

### Pre-Clinical Studies to Evaluate and Refine Imaging Performance

This Example optimizes technology for clinically-relevant 3-D contrast enhanced super-resolution imaging. To assess performance of the hardware, software, and algorithms developed in the Examples described herein above, imaging performance of the system is characterized both in phantoms and in-vivo. Imaging performance data is integrated iteratively with the development process.

Preliminary Data: Microvascular Imaging, Segmentation, and Analysis of Microvascular Morphology Metrics In Vivo.

We have conducted extensive in vivo imaging studies to date, in multiple rodent organs, and we have developed segmentation algorithms which enable extraction of microvessel data from 3-D Acoustic Angiography with collaborators at Kitware, Inc, [12, 14, 35, 48], as well as algorithms to quantify microvascular tortuosity, microvascular density, and vessel size distribution [1, 2, 12-14]. We have applied these analyses as a successful method of detecting unique microvascular “fingerprints” of spontaneous tumors. Receiver Operator Characteristic (ROC) curve analysis of data from 24 tumor bearing and 10 control mice indicated that the sum of angles tortuosity metric was superior for distinguishing tumor ROIs from controls, demonstrating sensitivity of 0.96 and specificity of 1.0 with an optimal threshold, based on microvascular data alone.[15] We expect this performance to be equivalent, if not better, with CESR, with the ability to resolve smaller vessels, and more significantly—we anticipate the increased penetration depth possible with CESR will make this technique even more clinically relevant.

Methods: In Vivo Testing and Analysis.

In vivo microvascular imaging is performed with two animal models. Initially, the rat FSA fibroscarcoma model is used. This rodent model is easy to prepare and produces a well vascularized subcutaneous tumor within two weeks, which has been studied extensively by our group and others.[1, 35, 36] It provides an “easy target” of in vivo tumor-associated angiogenesis for initial studies; however, it is not a good model for studying the very small vessels involved in tumor onset since it involves artificial implantation of cells (and these sub-100 micron vessels are a target of super-resolution). Thus, the C3Tag mouse mammary carcinoma model is also employed. This genetically engineered mouse model spontaneously develops breast cancer which mimics the human condition. The C3Tag has been extensively studied previously, and thus provides rigor and reproducibility to the animal imaging study [49-51]. Weekly imaging of these mice starting at 10 weeks enables imaging of tumor onset prior to when tumors are palpable, as we have described previously [2]. This model is an excellent assessment for our ability to image early microvascular abnormalities associated with very small tumors.

Methods: Benchmark Validation with Optical Imaging.

An existing validated method for angiogenesis measurement in vivo is the histologic measurement of microvessel density (MVD) [52]. Measurements of increased vessel density (hotspots) from biopsy specimens or excised tumors are believed to be useful in predicting metastasis risk, tumor growth and recurrence [52, 53, 54]. In order to benchmark our ability to visualize the smallest vessels in vivo with CESR, we visualize and quantify microvascular angiogenesis using 3-D stacking high-resolution microscopy [55, 56]. We compare optically measured (gold standard) microvessel diameter distributions vs. observations with CESR from tumor (and control tissue) samples from the aforementioned animal models.

Methods: Implementing Code for Tortuosity Metrics on Super-Resolution Data.

For CESR, three challenges exist for vessel segmentation and morphology characterization. For the first challenge, seed point selection is automated for the vessel imaging characteristics of the super-resolution images. The large number of vessels expected in these images requires automation. To address this challenge, vessel seed point selection is accomplished using machine learning methods that are trained using vessels that were manually seeded in the data. In particular, the extracted vessel centerlines from the manual seeds serve as positive cases for training a random forest classifier using feature vectors composed of local image intensity, derivative, and Hessian-based measures at multiple scales, as are used in automated vessel enhancement methods [57, 58]. Feature vectors from image points adjacent to the extracted vessels serve as negative training cases. The trained machine learning method is then used to estimate the likelihood ratio for points in new images, and points with high likelihood of being vessel points can be automatically chosen as seeds. For the second challenge, the traversal and radius estimation methods need to be updated for intensity profile of the super-resolution images. In working with Acoustic Angiography data in our prior studies we learned that intervening vessels can reduce the conspicuity of vessel segments that cross below them and that vessel profiles and noise require adapting the centerline and radii estimation processes. To address this challenge, the scale of the operators used is adapted to measure image intensity, derivative, and Hessian information during the traversal and radius estimation processes. This scaling is adapted during the experiments described below. Additionally, in the event that the vessel segments produced are incomplete, with a single vessel broken into multiple segments, post-processing the extracted vessel segments is performed: when the similarity of the locations and tangent directions of two segment ends suggest that they should be joined, the intervening space is probed in the seed-point likelihood ratio image, and if the likelihood ratio supports a vessel passing between those ends, then they will be joined into a larger, single vessel. In [59] this method was shown to perform as well as human experts in determining vessel segment connectivity. For the third challenge, all methods are ported to handle the huge CESR image volumes. The 3D CESR images can achieve 2048×2048×2048 voxels which occupies 16 gigabytes of memory if 16-bit values are stored.

Images are partitioned into overlapping regions that occupy 4 gigabytes of memory; methods are porting to use FFT-base scaled intensity, derivative, and Hessian measures that can be computed on GPUs via the ArrayFire algorithm, for these smaller images; and graphs merging methods are implemented to join the vascular trees from these overlapping regional images to recreate the complete 3D vascular tree of the full image. These analysis methods are ported over to a computational system. The same vessel density, branch frequency, and tortuosity measures from [1, 2, 60] our prior studies are applied to characterize the vascular network morphology and distinguishes cases of interest. All of the above methods are optimized using animal models as described elsewhere herein.

Methods: In Vitro System Characterization.

Micro-tubes ranging from 25 to 200 microns with precise separation distance (as assessed with a calibrated optical microscope) are utilized to characterize axial and lateral imaging performance of the imaging system, as well as to optimize pulsing parameters, and data processing. The goal of this optimization process is to select parameters (transmit frequency, pulse length, bubble concentration, and data processing parameters) to achieve the best resolution possible at 2-5 cm of penetration depth in tissue (or tissue equivalent), our target depth for clinical breast imaging, while maintaining sufficient contrast-to-tissue ratio such that our segmentation algorithms can extract microvascular features.

Methods: In vivo system assessment. Although phantom imaging is useful to determine optimal imaging parameters, CESR imaging with the prototype 3D super-resolution imaging system is conducted on rodents to verify imaging performance and perform final refinement of parameters before patient imaging. Tumor bearing animals are imaged using the imaging system described herein above. Tissue-mimicking phantom standoffs are used to vary spacing between the transducer and the subcutaneous tumor, with the goal of assessing in-vivo imaging performance at depths from 2-5 cm. Although ideally imaging parameters are optimized in the in vitro study, transmit pulse length, acoustic pressure, apodization, and receive filtering can be further optimization based on in vivo results.

Segmentation and analysis techniques as used in analysis of Acoustic Angiography data [1, 2, 12, 14] are employed. Nevertheless, CESR images are much larger than Acoustic Angiography images, and in the event that automated seed point selection fails, additional features such as measures of local contrast and responses from oriented match filters can be considered. In the worst case, the system can revert to using manual seed point selection. In the event that vessel imaging appearance is insufficient for traversal or radius estimation, the acquisition and bubble parameters can be adjusted; and in the worst case, the path minimization method of Frangi can be applied [58] which requires more labor but is much less sensitive to image noise.

### Example 11

### Pilot Human Clinical Study

A pilot study is performed in the human breast and thyroid to benchmark specificity to malignant lesions vs. standard of care grayscale ultrasound.

Methods.

The translation of super-resolution imaging into the clinic allows the practical assessment of the novel technology under real world conditions. We have selected two applications where over-diagnosis and the lack of specificity in conventional imaging approaches leads to over-diagnosis and the patient anxiety and potential risk of unnecessary biopsies. With conventional breast US and mammography, though highly sensitive for malignant lesions, the modalities lack specificity. Thyroid nodules are highly prevalent and identified on cross-sectional imaging and the workup of these lesions currently requires ultrasound guided biopsy, the vast majority of which are normal. In both applications, ultrasound is used as a part of the clinical workup, and a significant increase in specificity would substantially improve patient care, reduce unnecessary biopsies and reduce patient anxiety. From a pathophysiological perspective, the presence of increased microvasculature represents an increased risk of malignancy for both forms of cancer.[61, 62] Thus, this Example relates to pilot clinical imaging studies initially in volunteers to evaluate the imaging methodology, followed by preliminary observational studies in patients with known breast and thyroid lesions.

Patient Imaging.

All studies are approved by the Institutional Review Board (IRB) at the University of North Carolina at Chapel Hill, Chapel Hill, N.C., United States of America. Prior to any clinical imaging, the imaging system safety is assessed to ensure compliance with the Food and Drug Administration's (FDA) mechanical index (MI) and Thermal Index (TI) guidelines for diagnostic ultrasound imaging. Additionally, a mechanical positioning system with transducer mount is fixed to the ultrasound transducer to minimize motion and reduce operator fatigue. We have previously demonstrated success with this assisted positioning in other contrast ultrasound exams [63].

Initially, five volunteers are recruited for system imaging optimization. Subsequently two groups of patients, women undergoing core needle biopsy or surgical biopsy of breast lesions (BIRADS 4 and 5 breast lesions) and patients undergoing needle biopsy of thyroid lesions, are recruited. Patients are recruited through coordination with the UNC Radiology Clinics. Fifteen subjects are recruited for each anatomic region (thyroid or breast). In both cases, the pathological diagnosis serve as the reference standard for the study. Super Resolution imaging is performed in conjunction with standard diagnostic imaging, including b-mode ultrasound and Color Doppler. At the time of imaging, a single dose of an FDA-approved lipid-shell microbubble contrast agent (DEFINITY™) is administered intravenously by trained medical personnel. Imaging is performed by sonographer trained in advanced ultrasound techniques. Total imaging time for each subject is estimated to be less than 15 minutes. All image data is de-identified and transferred for off-line analysis based on a study ID. The research images are not interpreted or analyzed for clinical decisions related to the patient.

Reader Study.

A reader study is performed after the completion of patient accrual to study lesion characteristics under CESR as compared to conventional b-mode ultrasound. In these pilot observational studies, the primary aim is to evaluate the imaging approach for application in these two organs. We also estimate the receiver operating characteristic (ROC) curve for the CESR system. A total of five readers who are not investigators on this Example (for scientific rigor) (radiologists trained in breast imaging or ultrasound imaging) are recruited to participate for each reader study. The readers are asked to assign a probability score (1 to 5) and confidence for each lesion for each modality (0 to 100%). ROC analysis is performed as the primary analysis for the first aim. To compare the results from the two imaging modalities, we adopt the mixed effect ANOVA based on the Dorfman-Berbaum-Metz method. The outcome variable is the Tukey's jackknife pseudovalues of the AUCs from each reader and each patient under either modality, and separately for each anatomic region. The fixed effect in the independent list corresponds to the difference between the two modalities and the random effects will be used to account for within-patient and within-reader's correlations.

Possible interactions between the modalities and the readers and the patients are also included and tested for statistical significance. To test the main hypothesis, F-test statistic from the model parameter estimates is used to compare the mean AUCs between the b-mode ultrasound images and the CESR ultrasound images only.

Statistical Analysis.

For each lesion type, we assume 8 malignant and 7 benign and the AUC for conventional imaging to be 0.7. The power to detect 0.2 AUC difference is about 30%. However, our primary goal is to evaluate the technical ability of the CESR approach to examine lesions in each of these superficial anatomic regions. We also evaluate the number of vessels visualized, the effects of respiratory motion and radiologist confidence in interpreting the CESR microvascular images.

### Example 12

### Transcranial Super-Resolution Imaging Methods

This Example pertains to a paradigm-shifting approach for ultrasound imaging, which is normally not considered a feasible imaging modality in the brain due to heavy aberration from the skull and poor resolution from the low frequencies required to penetrate the skull. With the development of adaptive-beamforming super-resolution imaging, we are surprisingly able to correct for skull-induced aberration, and use super-resolution techniques to resolve brain microvasculature on the order of hundreds of microns, as well as assess local blood flow, even at low frequencies and several centimeters (˜8 cm) of depth.

Transcranial Focusing and Aberration Correction with Time Reversal Acoustics:

Propagation across the skull has been a persistent challenge for ultrasound because it 1) distorts the wave profile, which reduces resolution, and 2) it adds reverberation, which reduces contrast. Furthermore this distortion is linked to the skull morphology which varies significantly from person to person. In our previous work we have developed methods based on time reversal acoustics and highly accurate acoustic simulations of propagation through the human skull to correct for individual skull morphology. [64, 65]. These techniques were used successfully in non-invasive transcranial focused ultrasound surgery. The preliminary results in this Example show how time reversal phase correction techniques can be applied to detect contrast agents for transcranial super-resolution ultrasound imaging. This key innovation is necessary because the small quantity of contrast agents in microvessels would otherwise be undetectable due to reverberation and they would no longer be super-resolved due to the degradation in focusing quality.

This Example provides for improved super-resolution bubble detection and improved sensitivity transcranially and at depth in the brain, while preserving high frame rates; adaption of motion correction algorithms to our imaging approach; an improved method of obtaining velocity vector information; and evaluation bubble parameters for maximum performance.

Preliminary Data—Time Reversal Acoustics for Transcranial Phase Correction of Super-Resolution Imaging Sequences.

To date, all super-resolution contrast imaging is performed with plane wave imaging. This technique is fast, but has poor sensitivity as it uses low energy unfocused beams. It is therefore challenged to image clinically relevant concentrations of contrast agents at depth because it is severely degraded by the aberration and reverberation clutter introduced by the human skull. Even in the rat skull, which is much less distorting than the human skull, significant thinning was required to generate viable images in Errico's Nature Paper [5]. A method and simulation tools have been developed to correct for individual skull morphology and to restore contrast agent detectability for transcranial super-resolution imaging sequences).

Preliminary studies have compared the presently disclosed novel transcranial phase correction method with standard plane wave imaging, which is currently used for super-resolution sequences, and with conventional focused wave imaging, which is used in standard B-mode ultrasound. A juvenile porcine skull with a thickness between 1-3 mm (which is comparable to the temporal bone thickness in the human skull) was immersed in degassed water. A thin-walled microtube (Paradigm Optics Inc., Washington, United States of America) with an inner diameter of 150 μm was placed at a depth of 78 mm. Lipid-shelled microbubbles were pumped through the microtube, with the aid of an infusion pump (Harvard Apparatus, Holliston, Mass., United States of America) at 25 μL/min. Imaging was performed using a Verasonics Vantage system (Verasonics Inc., Redmond, Wash., United States of America) with a an ATL-Philips P4-1 probe, operating at 50 dB dynamic range. The transmitted pulses were 2 cycle sinusoids at 2.5 MHz.

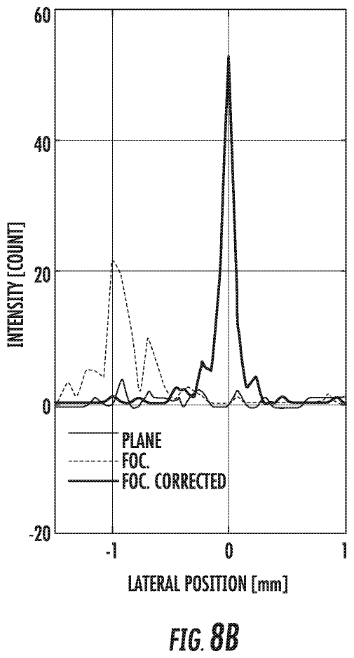

The conventional focused B-mode image (FIG. 8a) shows the skull at a depth between 12 and 20 mm. The 200 μm diameter target microtube was oriented orthogonally to the imaging plane and placed at 78 mm depth and 0 mm laterally. It appears to be over 30 mm wide and over 2 mm tall even though its cross section is orders of magnitude smaller due to aberration from the skull. Note that in between the skull and the microtube the B-mode image has a significant reverberation artefact.

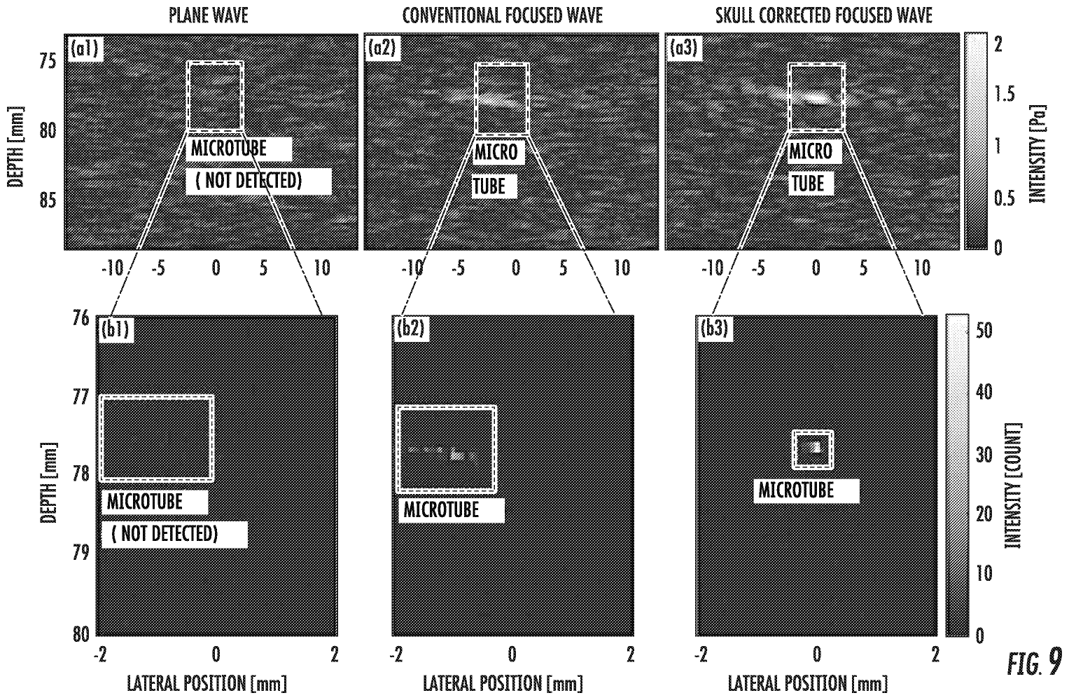

Subtraction images, which are used to detect contrast agent motion, show that plane wave imaging cannot detect microbubble motion (FIG. 9a1) and that the focused wave (FIG. 9a2) and adaptively-corrected focused wave (FIG. 9a3) can both detect motion. However, the zoomed in super-resolution image derived from a conventionally focused wave (FIG. 9b2) shows a microtube width of 1100 microns whereas the adaptively-corrected focused wave (FIG. 9b3) accurately determines a microtube width and height of 120 microns. Furthermore the microtube appears at the correct centered lateral position, whereas in the conventional focused wave the position error is >1 mm. The intensity for the corrected focused wave is also 2.5 times larger than in the conventional focused case. Therefore, the presently disclosed phase corrected transcranial super-resolution method can resolve a 150 micron tube at a clinically relevant 78 mm depth.

### Example 13

### Blood Flow Imaging Methods

Disclosed herein are novel imaging techniques implemented in hardware and software that provide unprecedented resolution for acoustic imaging. The following Example provides hardware and software approaches for a human brain imaging approach, referred to broadly as transcranial contrast enhanced super-resolution imaging (TCESR). THE TCESR approach provides for the visualization of microvessel structure and function in the human brain with ultrasound.

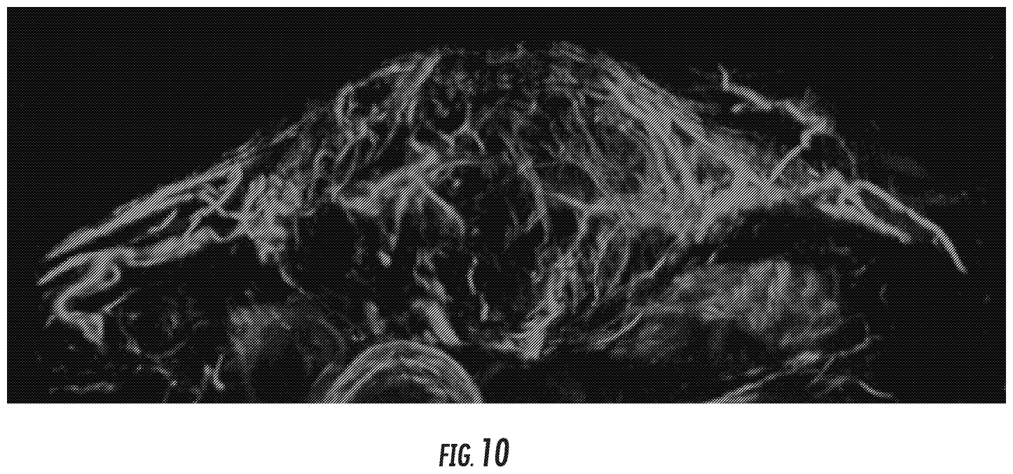

Recently, the revolutionary technology of localization microscopy (E. Betzig, et al., Science, vol. 313, pp. 1642-5, Sep. 15, 2006; M. J. Rust, et al., Nat Methods, vol. 3, pp. 793-5, October 2006) in the optical imaging domain has been translated into the medical ultrasound domain (C. Errico, et al., Nature, vol. 527, pp. 499-502, Nov. 26, 2015; F. Lin, et al., Theranostics, vol. 7, pp. 196-204, 2016). The ultrasound approach involves localizing the centers of detected contrast agents, and is referred to as ultrasound localization microscopy or contrast enhanced super-resolution (CESR) imaging. This technique enables imaging of microvessels at resolutions as small as ten micrometers, over an order of magnitude smaller than the ultrasound diffraction limit, and at depths much greater than possible with high frequency ultrasound. In a seminal Nature paper by Errico et al in 2015, investigators demonstrated the feasibility of contrast enhanced super resolution to image microvessels on the order of 10 microns in 2D planes of a rat brain through a thinned skull (C. Errico, et al., Nature, vol. 527, pp. 499-502, Nov. 26, 2015). Other techniques address the translational challenges for transcranial contrast enhanced super-resolution (TCESR) imaging in humans where the skull plays a determining acoustic role. These techniques are related to focused ultrasound surgery, which uses time-reversal acoustics to correct for the individual human skull morphology and to accurately focus ultrasound deep within brain (M. Fink, IEEE Trans Ultrason Ferroelectr Freq Control, vol. 39, pp. 555-66, 1992; G. F. Pinton, et al., IEEE Trans Ultrason Ferroelectr Freq Control, vol. 59, pp. 1149-59, June 2012). Super-resolution approaches provide for visualization of the microvasculature for imaging targets such as cancer associated angiogenesis in-vivo (FIG. 10) (F. Lin, et al., Theranostics, vol. 7, pp. 196-204, 2016). This technology, supported by ultra-fast imaging hardware and software capabilities, provides an innovative approach to transcranial imaging of the microvasculature deep within the brain.

Thus, an aspect of the presently disclosed subject matter provides an approach for ultrasound imaging, which is normally not considered a feasible imaging modality in the brain due to heavy aberration from the skull and poor resolution from the low frequencies required to penetrate the skull. With the development of adaptive-beamforming super-resolution imaging (described elsewhere herein), skull-induced aberrations are corrected for, and super-resolution techniques are used to resolve brain microvasculature on the order of hundreds of microns. Local blood flow is assessed, even at low frequencies and several centimeters (˜8 cm) of depth.

The following Example validates and characterizes blood flow estimation from transcranial contrast enhanced super-resolution ultrasound imaging using 9.4T MRI. A custom RF shielded ultrasound transducer acquires three-dimensional super-resolution flow data simultaneously with gold standard high resolution continuous arterial spin labeling perfusion MRI in an in vivo rat. Using a rat model of inhaled CO₂, which is known to modulate brain blood flow, sensitivity to blood flow changes in the rat brain is compared between the presently disclosed ultrasound technique and MRI.

The following Example shows that TCESR techniques provide high-resolution quantification of blood flow in vessels smaller than 200 microns at several centimeters in depth. The following Example also validates and characterizes blood flood estimation from TCESR by using a perfusion MR imaging as a gold standard reference. A custom MRI compatible ultrasound transducer is used to obtain near-simultaneous blood flow estimates in the in vivo rat. Super-resolution transcranial imaging approaches allow for the assessments changes in blood flow in the brain with a temporal and spatial resolution at least as good as (if not better than) high-field MRI arterial spin labeling, the current gold standard for brain blood flow imaging.

This Example shows that an ultrasound technology that resolves blood flow velocity at resolutions below 200 microns at 3 cm of depth. Particularly, this Example provides a super-resolution approach that resolves the velocity profile within a 200 micron vessel, at 5.8 cm depth. The observations of this Example extend into the brain using transcranial correction algorithms described elsewhere herein.

This Example extends the capabilities of ultrasound in terms of resolution and ability to acquire volumetric blood flow data so that the spatial resolution characteristics match (or exceed) those of MRI while retaining ultrasound's high temporal sampling capabilities. By cross-validating this technique with perfusion MRI the vast field of high resolution blood flow imaging and functional MRI becomes accessible to ultrasound. The intrinsic portability and low-cost advantages of ultrasound means that imaging can be performed directly in animal facilities, and that that animals can be awake and freely-moving during imaging (removing the need for anesthesia) (Sieu L-A, et al., Nat Methods 2015, 12:831-834). Furthermore, the increase in temporal resolution, which can go up to 10,000 volumetric acquisition per second, provides other applications that are inaccessible to MRI.

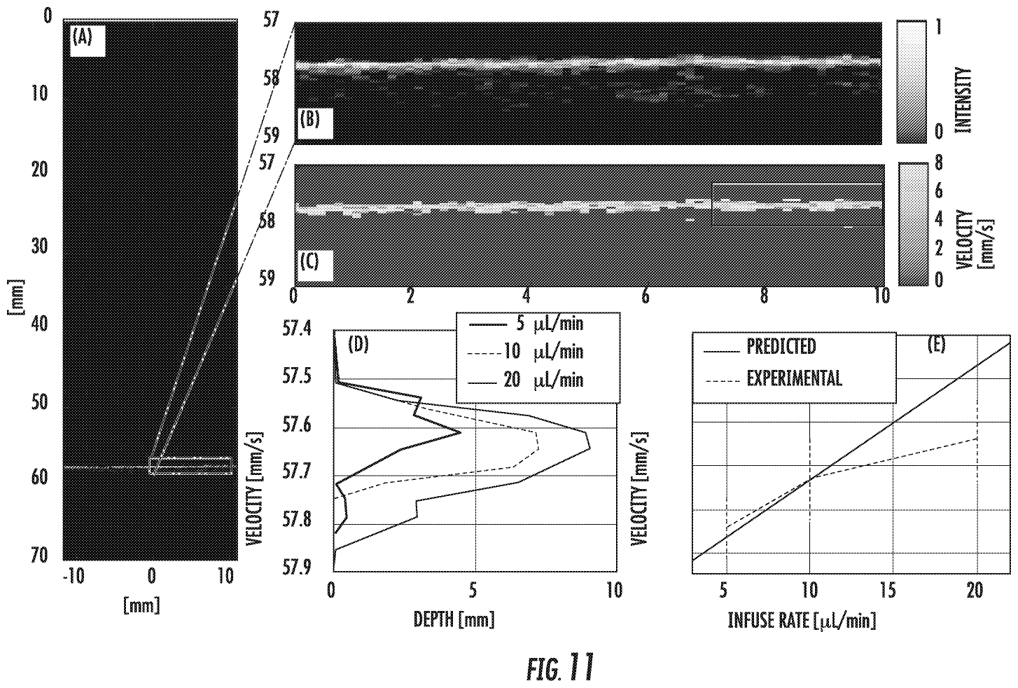

This Example compares TCESR-based blood flow imaging with near simultaneous MRI. To compare these modalities in the same animals, a blood flow method in the rat brain is provided, using a custom 1024 channel high frequency MRI compatible ultrasound transducer designed to operate at frequencies that are optimized for the rodent brain. Particularly, TCESR blood velocity estimation was acquired in a 200 micron diameter tube that was placed at a 57.7 mm depth from a standard diagnostic L7-4 ultrasound transducer. Microbubble infused water was then flowed through the tube using a computer-controlled injection pump at three rates 5, 10 and 20 microliters per minute. Then the microbubbles were tracked using the proposed TCESR velocity estimation method to generate a super-resolved image (FIG. 11a,b). The velocity, calculated by tracking the bubble centers, can be estimated along the microtube length (FIG. 11c) and the average measured velocity closely matches theoretical predictions based on conservation of mass (FIG. 11e). The measured velocity within the microtube was quantified across the diameter (FIG. 11d) demonstrating that a characteristic parabolic velocity profile can be measured inside the 200 micron tube at a depth of 58 mm. This suggests that the proposed TCESR velocity estimation technique will offer unprecedented spatio-temporal resolution of cerebral hemodynamics.

Experimental Methods:

The middle cerebral artery, which has a 400 micron diameter in the rat, is targeted because it is resolvable by MRI and super-resolution ultrasound. Furthermore, it is in the center of the brain, which simplifies targeting considerations for the ultrasound transducer. A total of 8 Long-Evans rats of both sexes, four female and four male, is used (a power analysis is included in the vertebrate animals section). A standard CO₂ inhalation protocol which modulates brain blood flow in the rat is used (Shih, Yen-Yu I., et al., Journal of Magnetic Resonance Imaging 40.3 (2014): 609-615).

Anesthetized rats are positioned in the MRI system in a custom holder device, which also maintains connection of the custom ultrasound transducer with the rat skull. Standard ultrasound imaging is first used for anatomical registration and then the transducer is fixed in place. The inhaled CO₂ protocol interleaves 5 minutes of baseline measurements with a varying amount of CO₂ premixed in air, which is used to induce changes in brain blood flow. Specifically, 1%, 0%, 3%, 0%, 5%, and 0% of CO₂ are administered with medical air, with 1 minute on and 5 minutes off in each case. Data are acquired with ultrasound, and with MRI, which are then used to compare TCESR blood flow estimates with MRI perfusion measurements using a correlation-based analysis.

Ultrasound Protocol:

As described elsewhere herein, transcranial ultrasound imaging utilizes a custom adaptive beamforming approach, implemented on a programmable ultrasound scanner. The scanner is a custom-designed Verasonics system, which can drive up to 1024 channels, and is upgraded with extreme high performance memory and parallel processing capability to perform the high-frame rate acquisition and processing needed for volumetric 3D adaptive multifocus imaging. The transducer is custom built for this project for MRI compatibility, and a higher frequency bandwidth (5-8 MHz) than the transducer for imaging through the human skull (1-2 MHz) described in Example 12. The higher frequency enables high resolution imaging in the rat brain. The ultrasound scanner is located outside of the MRI room and the transducer travels through the room bore hole. The transducer cable is shielded with braided wire and the transducer body is shielded with thin aluminum foil (C. Ma, et al., Biomedical Physics & Engineering Express, 2(4):047003, 2016). Both are grounded to the room. This method has been shown to result in no clinically relevant difference in ultrasound image quality (C. Ma, et al., Biomedical Physics & Engineering Express, 2(4):047003, 2016).

The imaging protocol is based on a time reversal transcranial phase correction method that accurately refocuses an ultrasound wave as it propagates through a non-uniform skull and that accounts for individual variations in skull morphology. This correction is applied twice, first when the wave propagates through the skull after being transmitted by the ultrasound probe, and then when the echo wave propagates from the target deep in the brain back to the probe. The phase correction is calculated with the FULLWAVE™ simulation tool (G. F. Pinton, et al., in IEEE Transactions on Ultrasonics, Ferroelectrics, and Frequency Control, vol. 56, no. 3, pp. 474-488, March 2009), an acoustic propagation method that can model the hard and soft tissue of the human body with high accuracy. Then, the adaptive multifocus method, which combines the advantages of the imaging quality of a focused transmission with the high frame-rates of a plane wave transmission, is used to simultaneously target multiple bubbles with a single emission. The raw 1024 channel RF data acquired with this ultrasound imaging sequence is used in super-resolution processing and analysis to detect microbubble velocities.

Ultrasound Processing and Analysis:

As demonstrated in the preliminary data, the bubble velocity vector is utilized to detect microvascular flow (C. Tremblay-Darveau, et al., IEEE Trans Med Imaging, vol. 35, pp. 699-709, February 2016). This approach provides flow vectors which can be utilized to provide additional quantitative data regarding microvessel pattern, for example, fluctuations in tumor blood flow which are known to cause hypoxia and re-oxygenation affecting tumor progression and response to treatment (L. I. Cardenas-Navia, et al., Cancer Res, vol. 68, pp. 5812-9, Jul. 15, 2008; H. Kimura, et al., Cancer Res, vol. 56, pp. 5522-8, Dec. 1, 1996). Flow vector mapping can also provide a separate tortuosity measurement for microvessels with opposite slow flow directions. This flow calculation method is based directly on the ability to accurately detect the position of single bubbles within the vessel. As a first step, the beamformed RF data from the ultrasound protocol undergoes a spatio-temporal filtering operation based on singular value decomposition, to separate bubble motion from background physiological motion. Then the bubble centers are super-localized using a point-spread-function deconvolution approach. Since the bubble position and frame rate is known, the velocity vector is extracted almost directly. Individual bubbles are tagged and velocity vectors are associated within each vessel. This data is aggregated into flow profiles along the length of each vessel (as demonstrated in FIG. 11). Rejection methods are investigated for confounding data, such as when two bubbles paths intersect.

MRI Protocol:

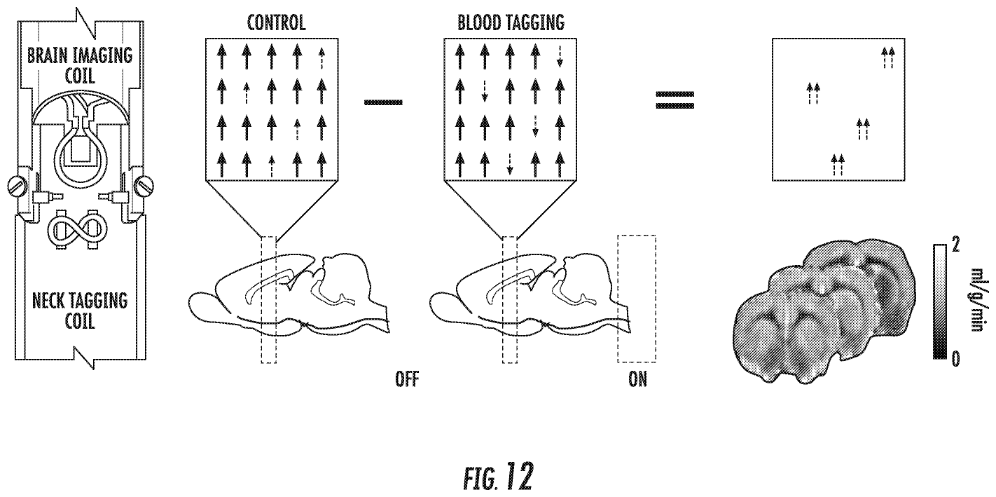

MRI is performed using a 9.4 Tesla Bruker BioSpec system with a BGA-9S gradient insert (Bruker Corp., Billerica, Mass., United States of America) at UNC Biomedical Research Imaging Center, Chapel Hill, N.C., United States of America. A home-made surface coil with an active decoupling circuit (internal diameter=2.0 cm) placed directly over the head is used as a transceiver and a separate butterfly-shaped coil underneath the neck is used for continuous arterial spin labeling (CASL), illustrated in FIG. 12. Magnetic field homogeneity has been previously optimized using standard FASTMAP shimming with first order shims on an isotropic voxel of 9×9×9 mm encompassing the imaging slices. A RARE T2-weighted pilot image is taken in the mid-sagittal plane to localize the anatomical position by identifying the anterior commissure at −0.36 mm posterior to bregma (Paxinos, G., Watson, C., 2014. The Rat Brain in Stereotaxic Coordinates. Elsevier Academic Press, Amsterdam; Boston). Cerebral blood flow (CBF) is measured by a validated CASL technique (Shih, Y. Y., et al., 2014a. Neurobiol Dis, 71, 131-139) using single shot gradient-echo echo-planar imaging (EPI) with bandwidth=250 kHz, TR/TE=3000/12 ms, labeling duration=2.4 s and post-labeling delay=250 ms, matrix=96×96, FOV=2.56×2.56 cm, 8 slices, and slice thickness=1 mm.

MRI Processing and Analysis:

Image analysis is performed using statistical parametric mapping (SPM) and a custom-written program in Matlab (MathWorks Inc., Natick, Mass., United States of America) (Shih, Y. Y., et al., 2008. J. Neurosci. Res. 86, 1801-1811). Skull stripping is performed manually with a threshold method. Automatic co-registration using SPM codes is used to correct slight image drift overtime within subjects. Previous results indicate that false movement will not occur after registration. Additionally, although the surface coil causes B1 inhomogeneity, this is empirically not corrected since it does not display significant effect when imaging the rat cortex (Lai, H. Y., et al., 2014. Neuroimage 84, 11-18; Lai, H. Y., et al., 2015. Magn. Reson. Med. 73 (3), 1246-51; Shih, Y. Y., et al., 2013. Neuroimage 73, 113-120; Shih, Y. Y., et al., 2014b. J. Cereb. Blood Flow Metab. 34 (9), 1483-92). CBF is calculated as: CBF=(Δ/T₁)(S_{C}−S_{L})/(S_{L}+(2α−1)S_{C}), where S_{C} and S_{L} are the MR signal intensities from the control and labeled images, respectively. λ is the water brain-blood partition coefficient, T₁ is that of tissue, and a is the arterial spin-labeling efficiency. Values of A, T₁, and α are set to 0.9 (Herscovitch, P., Raichle, M. E., 1985. J. Cereb. Blood Flow Metab. 5, 65-69) 1.9 s (de Graaf, R. A., et al., 2006. Magn. Reson. Med. 56, 386-394), and 0.7 (Shih, Y. Y., et al., 2011. J. Cereb. Blood Flow Metab. 31, 832-841), respectively. Pairwise subtraction is first performed between the control and labeled images, followed by a subsequent pairwise subtraction between the current labeled image and the next.

Comparison of MRI and US Data:

Quantitative CBF maps acquired at a 200×200 micron resolution with MRI and quantitative blood velocity estimates determined with TCESR ultrasound are compared at the location of the middle cerebral artery. A correlation analysis based on the perfusion (for MRI) or velocity (for ultrasound) estimates integrated over the volume middle cerebral artery and as a function of CO₂ concentration are used to establish the similarity between the two imaging methods. A sensitivity analysis based on the same integrated blood hemodynamics measurements characterizes the modalities' respective capabilities in detecting the onset of CO₂ induced vascular changes, i.e. to establish a noise floor.

Alternative Strategies:

The CO₂ challenge protocol might not target the ideal range hemodynamic and can be modified to according to the observed sensitivity (e.g. 1% increments instead of 2%, or different thing intervals). The near simultaneous MRI and Ultrasound may also present challenges since despite the MR compatibility of the custom transducer, MR artifacts are still possible due to PZT crystals in the probe. The stereotaxic transducer positioning system we have designed is 3D printed and thus allows rapid testing of multiple configurations to minimize gradient disruption. An MRI compatible translation stage designed to position ultrasound transducers (FUS Instruments LP100), which is currently housed in our small animal imaging facility, may also help in the determining the optimal design.

### REFERENCES

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.
[1] R. C. Gessner, S. R. Aylward, and P. A. Dayton, “Mapping microvasculature with acoustic angiography yields quantifiable differences between healthy and tumor-bearing tissue volumes in a rodent model,” Radiology, vol. 264, pp. 733-40, September 2012.
[2] S. E. Shelton, Y. Z. Lee, M. Lee, E. Cherin, F. S. Foster, S. R. Aylward, et al., “Quantification of Microvascular Tortuosity during Tumor Evolution Using Acoustic Angiography,” Ultrasound Med Biol, vol. 41, pp. 1896-904, July 2015.
[3] E. Betzig, G. H. Patterson, R. Sougrat, O. W. Lindwasser, S. Olenych, J. S. Bonifacino, et al., “Imaging intracellular fluorescent proteins at nanometer resolution,” Science, vol. 313, pp. 1642-5, Sep. 15, 2006.
[4] M. J. Rust, M. Bates, and X. Zhuang, “Sub-diffraction-limit imaging by stochastic optical reconstruction microscopy (STORM),” Nat Methods, vol. 3, pp. 793-5, October 2006.
[5] C. Errico, J. Pierre, S. Pezet, Y. Desailly, Z. Lenkei, O. Couture, et al., “Ultrafast ultrasound localization microscopy for deep super-resolution vascular imaging,” Nature, vol. 527, pp. 499-502, Nov. 26, 2015.
[6] F. Lin, S. E. Shelton, D. Espindola, J. D. Rojas, G. Pinton, and P. A. Dayton, “3-D Ultrasound Localization Microscopy for Identifying Microvascular Morphology Features of Tumor Angiogenesis at a Resolution Beyond the Diffraction Limit of Conventional Ultrasound” Theranostics, vol. 7, pp. 196-204, 2016.
[7] J. Folkman, “Tumor angiogenesis: therapeutic implications,” N Engl J Med, vol. 285, pp. 1182-6, Nov. 18, 1971.
[8] D. Hanahan and R. A. Weinberg, “The hallmarks of cancer,” Cell, vol. 100, pp. 57-70, Jan. 7, 2000.
[9] D. Hanahan and R. A. Weinberg, “Hallmarks of cancer: the next generation,” Cell, vol. 144, pp. 646-74, Mar. 4, 2011.
[10] C. Y. Li, S. Shan, Q. Huang, R. D. Braun, J. Lanzen, K. Hu, et al., “Initial stages of tumor cell-induced angiogenesis: evaluation via skin window chambers in rodent models,” J Natl Cancer Inst, vol. 92, pp. 143-7, Jan. 19, 2000.
[11] R. Gessner, M. Lukacs, M. Lee, E. Cherin, F. S. Foster, and P. A. Dayton, “High-resolution, high-contrast ultrasound imaging using a prototype dual-frequency transducer: in vitro and in vivo studies,” IEEE Trans Ultrason Ferroelectr Freq Control, vol. 57, pp. 1772-81, August 2010.
[12] R. C. Gessner, C. B. Frederick, F. S. Foster, and P. A. Dayton, “Acoustic angiography: a new imaging modality for assessing microvasculature architecture,” Int J Biomed Imaging, vol. 2013, p. 936593, 2013.
[13] J. M. Dunleavey, L. Xiao, J. Thompson, M. M. Kim, J. M. Shields, S. E. Shelton, et al., “Vascular channels formed by subpopulations of PECAM1+ melanoma cells,” Nat Commun, vol. 5, p. 5200, Oct. 22, 2014.
[14] S. R. Rao, S. E. Shelton, and P. A. Dayton, “The “Fingerprint” of Cancer Extends Beyond Solid Tumor Boundaries: Assessment With a Novel Ultrasound Imaging Approach,” IEEE Trans Biomed Eng, vol. 63, pp. 1082-6, May 2016.
[15] S. E. Shelton, S. Aylward, F. S. Foster, and P. A. Dayton, “The Application of Acoustic Angiography to Assess the Progression of Angiogenesis in a Spontaneous Mouse Model of Breast Cancer,” in IEEE Ultrasonics Symposium, Tours, France, 2016.
[16] K. Christensen-Jeffries, R. J. Browning, M. X. Tang, C. Dunsby, and R. J. Eckersley, “In vivo acoustic superresolution and super-resolved velocity mapping using microbubbles,” IEEE Trans Med Imaging, vol. 34, pp. 433-40, February 2015.
[17] M. A. O'Reilly and K. Hynynen, “A super-resolution ultrasound method for brain vascular mapping,” Med Phys, vol. 40, p. 110701, November 2013.
[18] Y. Desailly, O. Couture, M. Fink, and M. Tanter, “Sono-activated ultrasound localization microscopy,” Applied Physics Letters, vol. 103, Oct. 21, 2013.
[19] S. S. Abdelmoneim, M. Bernier, C. G. Scott, A. Dhoble, S. A. Ness, M. E. Hagen, et al., “Safety of contrast agent use during stress echocardiography: a 4-year experience from a single-center cohort study of 26,774 patients,” JACC Cardiovasc Imaging, vol. 2, pp. 1048-56, September 2009.
[20] S. S. Abdelmoneim, M. Bernier, C. G. Scott, A. Dhoble, S. A. Ness, M. E. Hagen, et al., “Safety of contrast agent use during stress echocardiography in patients with elevated right ventricular systolic pressure: a cohort study,” Circ Cardiovasc Imaging, vol. 3, pp. 240-8, May 2010.
[21] M. S. Dolan, S. S. Gala, S. Dodla, S. S. Abdelmoneim, F. Xie, D. Cloutier, et al., “Safety and efficacy of commercially available ultrasound contrast agents for rest and stress echocardiography a multicenter experience,” J Am Coll Cardiol, vol. 53, pp. 32-8, Jan. 6, 2009.
[22] S. L. Mulvagh, “Safety of Ultrasound Contrast Studies,” in Diagnostic Ultrasound Annual Conference, 2010
[23] J. Holash, P. C. Maisonpierre, D. Compton, P. Boland, C. R. Alexander, D. Zagzag, et al., “Vessel cooption, regression, and growth in tumors mediated by angiopoietins and VEGF,” Science, vol. 284, pp. 1994-8, Jun. 18, 1999.
[24] J. Holash, S. J. Wiegand, and G. D. Yancopoulos, “New model of tumor angiogenesis: dynamic balance between vessel regression and growth mediated by angiopoietins and VEGF,” Oncogene, vol. 18, pp. 5356-62, Sep. 20, 1999.
[25] G. D. Yancopoulos, S. Davis, N. W. Gale, J. S. Rudge, S. J. Wiegand, and J. Holash, “Vascular-specific growth factors and blood vessel formation,” Nature, vol. 407, pp. 242-8, Sep. 14, 2000.
[26] D. Hanahan and J. Folkman, “Patterns and emerging mechanisms of the angiogenic switch during tumorigenesis,” Cell, vol. 86, pp. 353-64, Aug. 9, 1996.
[27] J. Folkman, “Angiogenesis,” Annu Rev Med, vol. 57, pp. 1-18, 2006.
[28] J. A. Nagy, S. H. Chang, S. C. Shih, A. M. Dvorak, and H. F. Dvorak, “Heterogeneity of the tumor vasculature,” Semin Thromb Hemost, vol. 36, pp. 321-31, April 2010.
[29] P. Baluk, H. Hashizume, and D. M. McDonald, “Cellular abnormalities of blood vessels as targets in cancer,” Curr Opin Genet Dev, vol. 15, pp. 102-11, February 2005.
[30] J. W. Baish and R. K. Jain, “Fractals and cancer,” Cancer Res, vol. 60, pp. 3683-8, Jul. 15, 2000.
[31] E. Bullitt, M. G. Ewend, S. Aylward, W. Lin, G. Gerig, S. Joshi, et al., “Abnormal vessel tortuosity as a marker of treatment response of malignant gliomas: preliminary report,” Technol Cancer Res Treat, vol. 3, pp. 577-84, December 2004.
[32] E. Bullitt, N. U. Lin, M. G. Ewend, D. Zeng, E. P. Winer, L. A. Carey, et al., “Tumor therapeutic response and vessel tortuosity: preliminary report in metastatic breast cancer,” Med Image Comput Comput Assist Interv, vol. 9, pp. 561-8, 2006.
[33] S. Kasoji, J. Rivera, R. Gessner, X. S. Chang, and P. A. Dayton, “Early assessment of tumor response to radiation therapy using high-resolution quantitative microvascular imaging,” in IEEE Ultrasonics Symposium, Tours, France, 2016.
[34] C. Demene, T. Deffieux, M. Pernot, B. F. Osmanski, V. Biran, J. L. Gennisson, et al., “Spatiotemporal Clutter Filtering of Ultrafast Ultrasound Data Highly Increases Doppler and fUltrasound Sensitivity,” IEEE Trans Med Imaging, vol. 34, pp. 2271-85, November 2015.
[35] S. E. Shelton, B. D. Lindsey, J. K. Tsuruta, F. S. Foster, and P. A. Dayton, “Molecular Acoustic Angiography: A New Technique for High-resolution Superharmonic Ultrasound Molecular Imaging,” Ultrasound Med Biol, vol. 42, pp. 769-81, March 2016.
[36] J. E. Streeter, R. Gessner, I. Miles, and P. A. Dayton, “Improving sensitivity in ultrasound molecular imaging by tailoring contrast agent size distribution: in vivo studies,” Mol Imaging, vol. 9, pp. 87-95, April 2010.
[37] G. F. Pinton, J. Dahl, S. Rosenzweig, and G. E. Trahey, “A heterogeneous nonlinear attenuating full-wave model of ultrasound,” IEEE Trans Ultrason Ferroelectr Freq Control, vol. 56, pp. 474-88, March 2009.
[38] G. F. Pinton, G. E. Trahey, and J. J. Dahl, “Sources of image degradation in fundamental and harmonic ultrasound imaging using nonlinear, full-wave simulations,” IEEE Trans Ultrason Ferroelectr Freq Control, vol. 58, pp. 754-65, April 2011.
[39] M. Tanter, J. Bercoff, L. Sandrin, and M. Fink, “Ultrafast compound imaging for 2-D motion vector estimation: application to transient elastography,” IEEE Trans Ultrason Ferroelectr Freq Control, vol. 49, pp. 1363-74, October 2002.
[40] B. Denarie, T. A. Tangen, I. K. Ekroll, N. Rolim, H. Torp, T. Bjastad, et al., “Coherent plane wave compounding for very high frame rate ultrasonography of rapidly moving targets,” IEEE Trans Med Imaging, vol. 32, pp. 1265-76, July 2013.
[41] A. Bar-Zion, C. Tremblay-Darveau, O. Solomon, D. Adam, and Y. Eldar, “Fast Vascular Ultrasound Imaging with Enhanced Spatial Resolution and Background Rejection,” IEEE Trans Med Imaging, Aug. 15, 2016.
[42] BraccoDiagnostics, “Lumason prescribing information,” ed, 2014.
[43] LantheusMedicallmaging, “Prescribing Information for Definity,” ed, 2016.
[44] GEHealthcare, “Optison prescribing information,” ed, 2012.
[45] C. Tremblay-Darveau, R. Williams, L. Milot, M. Bruce, and P. N. Burns, “Visualizing the Tumor Microvasculature With a Nonlinear Plane-Wave Doppler Imaging Scheme Based on Amplitude Modulation,” IEEE Trans Med Imaging, vol. 35, pp. 699-709, February 2016.
[46] L. I. Cardenas-Navia, D. Mace, R. A. Richardson, D. F. Wilson, S. Shan, and M. W. Dewhirst, “The pervasive presence of fluctuating oxygenation in tumors,” Cancer Res, vol. 68, pp. 5812-9, Jul. 15, 2008.
[47] H. Kimura, R. D. Braun, E. T. Ong, R. Hsu, T. W. Secomb, D. Papahadjopoulos, et al., “Fluctuations in red cell flux in tumor microvessels can lead to transient hypoxia and reoxygenation in tumor parenchyma,” Cancer Res, vol. 56, pp. 5522-8, Dec. 1, 1996.
[48] P. A. Dayton, R. C. Gessner, L. Phillips, S. E. Shelton, K. Heath Martin, M. Lee, et al., “The implementation of acoustic angiography for microvascular and angiogenesis imaging,” Conf Proc IEEE Eng Med Biol Soc, vol. 2014, pp. 4283-5, 2014.
[49] J. I. Herschkowitz, X. He, C. Fan, and C. M. Perou, “The functional loss of the retinoblastoma tumour suppressor is a common event in basal-like and luminal B breast carcinomas,” Breast Cancer Res, vol. 10, p. R75, 2008.
[50] J. I. Herschkowitz, K. Simin, V. J. Weigman, I. Mikaelian, J. Usary, Z. Hu, et al., “Identification of conserved gene expression features between murine mammary carcinoma models and human breast tumors,” Genome Biol, vol. 8, p. R76, 2007.
[51] I. G. Maroulakou, M. Anver, L. Garrett, and J. E. Green, “Prostate and mammary adenocarcinoma in transgenic mice carrying a rat C3(1) simian virus 40 large tumor antigen fusion gene,” Proc Natl Acad Sci USA, vol. 91, pp. 11236-40, Nov. 8, 1994.
[52] S. M. Stieger, S. H. Bloch, O. Foreman, E. R. Wisner, K. W. Ferrara, and P. A. Dayton, “Ultrasound assessment of angiogenesis in a matrigel model in rats,” Ultrasound Med Biol, vol. 32, pp. 673-81, May 2006.
[53] W. W. Choi, M. M. Lewis, D. Lawson, Q. Yin-Goen, G. G. Birdsong, G. A. Cotsonis, et al., “Angiogenic and lymphangiogenic microvessel density in breast carcinoma: correlation with clinicopathologic parameters and VEGF-family gene expression,” Mod Pathol, vol. 18, pp. 143-52, January 2005.
[54] J. B. Laforga and F. I. Aranda, “Angiogenic Index: A New Method for Assessing Microvascularity in Breast Carcinoma with Possible Prognostic Implications,” Breast J, vol. 6, pp. 103-107, March 2000.
[55] J. C. Chappell, J. G. Cluceru, J. E. Nesmith, K. P. Mouillesseaux, V. B. Bradley, C. M. Hartland, et al., “Flt-1 (VEGFR-1) coordinates discrete stages of blood vessel formation,” Cardiovasc Res, vol. 111, pp. 84-93, Jul. 1, 2016.
[56] S. M. Peirce, F. Mac Gabhann, and V. L. Bautch, “Integration of experimental and computational approaches to sprouting angiogenesis,” Curr Opin Hematol, vol. 19, pp. 184-91, May 2012.
[57] S. R. Aylward and E. Bullitt, “Initialization, noise, singularities, and scale in height ridge traversal for tubular object centerline extraction,” IEEE Trans Med Imaging, vol. 21, pp. 61-75, February 2002.
[58] A. F. Frangi, W. J. Niessen, K. L. Vincken, and M. A. Viergever, “Multiscale vessel enhancement filtering,” in MICCAI, 1998, pp. 130-137.
[59] E. Bullitt, S. Aylward, A. Liu, J. Stone, S. Mukherji, C. Coffey, et al., “3D graph description of the intracerebral vasculature from segmented MRA and tests of accuracy by comparison with X-ray angiograms,” in INFORMATION PROCESSING IN MEDICAL IMAGING, 1999, pp. 308-321.
[60] E. Bullitt, K. E. Muller, I. Jung, W. Lin, and S. Aylward, “Analyzing attributes of vessel populations,” Med Image Anal, vol. 9, pp. 39-49, February 2005.
[61] J. Jiang, X. Shang, H. Zhang, W. Ma, Y. Xu, Q. Zhou, et al., “Correlation between maximum intensity and microvessel density for differentiation of malignant from benign thyroid nodules on contrast-enhanced sonography,” J Ultrasound Med, vol. 33, pp. 1257-63, July 2014.
[62] C. A. Sullivan, S. Ghosh, I. T. Ocal, R. L. Camp, D. L. Rimm, and G. G. Chung, “Microvessel area using automated image analysis is reproducible and is associated with prognosis in breast cancer,” Hum Pathol, vol. 40, pp. 156-65, February 2009.
[63] S. K. Kasoji, E. H. Chang, L. B. Mullin, W. K. Chong, W. K. Rathmell, and P. A. Dayton, “A Pilot Clinical Study in Characterization of Malignant Renal Cell Carcinoma Subtype with Contrast-Enhanced Ultrasound,” Ultrason Imaging, Sep. 22, 2016.
[64] G. F. Pinton, J. F. Aubry, and M. Tanter, “Direct phase projection and transcranial focusing of ultrasound for brain therapy,” IEEE Trans Ultrason Ferroelectr Freq Control, vol. 59, pp. 1149-59, June 2012.
[65] G. Pinton, J. F. Aubry, M. Fink, and M. Tanter, “Effects of nonlinear ultrasound propagation on high intensity brain therapy,” Med Phys, vol. 38, pp. 1207-16, March 2011.
[66] E. Betzig, G. H. Patterson, R. Sougrat, O. W. Lindwasser, S. Olenych, J. S. Bonifacino, et al., “Imaging intracellular fluorescent proteins at nanometer resolution,” Science, vol. 313, pp. 1642-5, Sep. 15, 2006.
[67] M. J. Rust, M. Bates, and X. Zhuang, “Sub-diffraction-limit imaging by stochastic optical reconstruction microscopy (STORM),” Nat Methods, vol. 3, pp. 793-5, October 2006.
[68] F. Lin, S. E. Shelton, D. Espindola, J. D. Rojas, G. Pinton, and P. A. Dayton, “3-D Ultrasound Localization Microscopy for Identifying Microvascular Morphology Features of Tumor Angiogenesis at a Resolution Beyond the Diffraction Limit of Conventional Ultrasound” Theranostics, vol. 7, pp. 196-204, 2016.
[69] M. Fink, “Time reversal of ultrasonic fields. I. Basic principles,” IEEE Trans Ultrason Ferroelectr Freq Control, vol. 39, pp. 555-66, 1992.
[70] K. Christensen-Jeffries, R. J. Browning, M. X. Tang, C. Dunsby, and R. J. Eckersley, “In vivo acoustic super-resolution and super-resolved velocity mapping using microbubbles,” IEEE Trans Med Imaging, vol. 34, pp. 433-40, February 2015.
[71] M. A. O'Reilly and K. Hynynen, “A super-resolution ultrasound method for brain vascular mapping,” Med Phys, vol. 40, p. 110701, November 2013.
[72] Y. Desailly, O. Couture, M. Fink, and M. Tanter, “Sono-activated ultrasound localization microscopy,” Applied Physics Letters, vol. 103, Oct. 21, 2013.
[73] E. Mace, G. Montaldo, I. Cohen, M. Baulac, M. Fink, and M. Tanter, “Functional ultrasound imaging of the brain,” Nat Methods, vol. 8, pp. 662-4, Jul. 3, 2011.
[74] S. S. Abdelmoneim, M. Bernier, C. G. Scott, A. Dhoble, S. A. Ness, M. E. Hagen, et al., “Safety of contrast agent use during stress echocardiography: a 4-year experience from a single-center cohort study of 26,774 patients,” JACC Cardiovasc Imaging, vol. 2, pp. 1048-56, September 2009.
[75] S. S. Abdelmoneim, M. Bernier, C. G. Scott, A. Dhoble, S. A. Ness, M. E. Hagen, et al., “Safety of contrast agent use during stress echocardiography in patients with elevated right ventricular systolic pressure: a cohort study,” Circ Cardiovasc Imaging, vol. 3, pp. 240-8, May 2010.
[76] M. S. Dolan, S. S. Gala, S. Dodla, S. S. Abdelmoneim, F. Xie, D. Cloutier, et al., “Safety and efficacy of commercially available ultrasound contrast agents for rest and stress echocardiography a multicenter experience,” J Am Coll Cardiol, vol. 53, pp. 32-8, Jan. 6, 2009.
[77] S. L. Mulvagh, “Safety of Ultrasound Contrast Studies,” in Diagnostic Ultrasound Annual Conference, 2010.
[78] E. Betzig, G. H. Patterson, R. Sougrat, O. W. Lindwasser, S. Olenych, J. S. Bonifacino, et al., “Imaging intracellular fluorescent proteins at nanometer resolution,” Science, vol. 313, pp. 1642-5, Sep. 15, 2006.
[79] M. J. Rust, M. Bates, and X. Zhuang, “Sub-diffraction-limit imaging by stochastic optical reconstruction microscopy (STORM),” Nat Methods, vol. 3, pp. 793-5, October 2006.
[80] C. Errico, J. Pierre, S. Pezet, Y. Desailly, Z. Lenkei, O. Couture, et al., “Ultrafast ultrasound localization microscopy for deep super-resolution vascular imaging,” Nature, vol. 527, pp. 499-502, Nov. 26, 2015.
[81] F. Lin, S. E. Shelton, D. Espindola, J. D. Rojas, G. Pinton, and P. A. Dayton, “3-D Ultrasound Localization Microscopy for Identifying Microvascular Morphology Features of Tumor Angiogenesis at a Resolution Beyond the Diffraction Limit of Conventional Ultrasound” Theranostics, vol. 7, pp. 196-204, 2016.
[82] M. Fink, “Time reversal of ultrasonic fields. I. Basic principles,” IEEE Trans Ultrason Ferroelectr Freq Control, vol. 39, pp. 555-66, 1992.
[83] G. F. Pinton, J. F. Aubry, and M. Tanter, “Direct phase projection and transcranial focusing of ultrasound for brain therapy,” IEEE Trans Ultrason Ferroelectr Freq Control, vol. 59, pp. 1149-59, June 2012.
[84] K. Christensen-Jeffries, R. J. Browning, M. X. Tang, C. Dunsby, and R. J. Eckersley, “In vivo acoustic super-resolution and super-resolved velocity mapping using microbubbles,” IEEE Trans Med Imaging, vol. 34, pp. 433-40, February 2015.
[85] M. A. O'Reilly and K. Hynynen, “A super-resolution ultrasound method for brain vascular mapping,” Med Phys, vol. 40, p. 110701, November 2013.
[86] Y. Desailly, O. Couture, M. Fink, and M. Tanter, “Sono-activated ultrasound localization microscopy,” Applied Physics Letters, vol. 103, Oct. 21, 2013.
[87] E. Mace, G. Montaldo, I. Cohen, M. Baulac, M. Fink, and M. Tanter, “Functional ultrasound imaging of the brain,” Nat Methods, vol. 8, pp. 662-4, Jul. 3, 2011.
[88] S. S. Abdelmoneim, M. Bernier, C. G. Scott, A. Dhoble, S. A. Ness, M. E. Hagen, et al., “Safety of contrast agent use during stress echocardiography: a 4-year experience from a single-center cohort study of 26,774 patients,” JACC Cardiovasc Imaging, vol. 2, pp. 1048-56, September 2009.
[89] S. S. Abdelmoneim, M. Bernier, C. G. Scott, A. Dhoble, S. A. Ness, M. E. Hagen, et al., “Safety of contrast agent use during stress echocardiography in patients with elevated right ventricular systolic pressure: a cohort study,” Circ Cardiovasc Imaging, vol. 3, pp. 240-8, May 2010.
[90] M. S. Dolan, S. S. Gala, S. Dodla, S. S. Abdelmoneim, F. Xie, D. Cloutier, et al., “Safety and efficacy of commercially available ultrasound contrast agents for rest and stress echocardiography a multicenter experience,” J Am Coll Cardiol, vol. 53, pp. 32-8, Jan. 6, 2009.
[91] S. L. Mulvagh, “Safety of Ultrasound Contrast Studies,” in Diagnostic Ultrasound Annual Conference, 2010.
[92] G. Pinton, J. F. Aubry, M. Fink, and M. Tanter, “Effects of nonlinear ultrasound propagation on high intensity brain therapy,” Med Phys, vol. 38, pp. 1207-16, March 2011.
[93] C. Demene, T. Deffieux, M. Pernot, B. F. Osmanski, V. Biran, J. L. Gennisson, et al., “Spatiotemporal Clutter Filtering of Ultrafast Ultrasound Data Highly Increases Doppler and fUltrasound Sensitivity,” IEEE Trans Med Imaging, vol. 34, pp. 2271-85, November 2015.
[94] R. C. Gessner, C. B. Frederick, F. S. Foster, and P. A. Dayton, “Acoustic angiography: a new imaging modality for assessing microvasculature architecture,” Int J Biomed Imaging, vol. 2013, p. 936593, 2013.
[95] R. C. Gessner, S. R. Aylward, and P. A. Dayton, “Mapping microvasculature with acoustic angiography yields quantifiable differences between healthy and tumor-bearing tissue volumes in a rodent model,” Radiology, vol. 264, pp. 733-40, September 2012.
[96] S. E. Shelton, Y. Z. Lee, M. Lee, E. Cherin, F. S. Foster, S. R. Aylward, et al., “Quantification of Microvascular Tortuosity during Tumor Evolution Using Acoustic Angiography,” Ultrasound Med Biol, vol. 41, pp. 1896-904, July 2015.
[97] S. E. Shelton, B. D. Lindsey, J. K. Tsuruta, F. S. Foster, and P. A. Dayton, “Molecular Acoustic Angiography: A New Technique for High-resolution Superharmonic Ultrasound Molecular Imaging,” Ultrasound Med Biol, vol. 42, pp. 769-81, March 2016.
[98] J. E. Streeter, R. Gessner, I. Miles, and P. A. Dayton, “Improving sensitivity in ultrasound molecular imaging by tailoring contrast agent size distribution: in vivo studies,” Mol Imaging, vol. 9, pp. 87-95, April 2010.
[99] G. F. Pinton, J. Dahl, S. Rosenzweig, and G. E. Trahey, “A heterogeneous nonlinear attenuating full-wave model of ultrasound,” IEEE Trans Ultrason Ferroelectr Freq Control, vol. 56, pp. 474-88, March 2009.
[100] G. F. Pinton, G. E. Trahey, and J. J. Dahl, “Sources of image degradation in fundamental and harmonic ultrasound imaging using nonlinear, full-wave simulations,” IEEE Trans Ultrason Ferroelectr Freq Control, vol. 58, pp. 754-65, April 2011.
[101] BraccoDiagnostics, “Lumason prescribing information,” ed, 2014.
[102] LantheusMedicallmaging, “Prescribing Information for Definity,” ed, 2016.
[103] GEHealthcare, “Optison prescribing information,” ed, 2012.
[104] C. Tremblay-Darveau, R. Williams, L. Milot, M. Bruce, and P. N. Burns, “Visualizing the Tumor Microvasculature With a Nonlinear Plane-Wave Doppler Imaging Scheme Based on Amplitude Modulation,” IEEE Trans Med Imaging, vol. 35, pp. 699-709, February 2016.
[105] L. I. Cardenas-Navia, D. Mace, R. A. Richardson, D. F. Wilson, S. Shan, and M. W. Dewhirst, “The pervasive presence of fluctuating oxygenation in tumors,” Cancer Res, vol. 68, pp. 5812-9, Jul. 15, 2008.
[106] H. Kimura, R. D. Braun, E. T. Ong, R. Hsu, T. W. Secomb, D. Papahadjopoulos, et al., “Fluctuations in red cell flux in tumor microvessels can lead to transient hypoxia and reoxygenation in tumor parenchyma,” Cancer Res, vol. 56, pp. 5522-8, Dec. 1, 1996.
[107] B. D. Lindsey, H. A. Nicoletto, E. R. Bennett, D. T. Laskowitz, and S. W. Smith, “3-D transcranial ultrasound imaging with bilateral phase aberration correction of multiple isoplanatic patches: a pilot human study with microbubble contrast enhancement,” Ultrasound Med Biol, vol. 40, pp. 90-101, January 2014
[108] Sieu L-A, Bergel A, Tiran E, Deffieux T, Pernot M, Gennisson J-L, Tanter M, Cohen I: EEG and functional ultrasound imaging in mobile rats. Nat Methods 2015, 12:831-834.
[109] Shih, Yen-Yu I., et al. “Comparison of retinal and cerebral blood flow between continuous arterial spin labeling MRI and fluorescent microsphere techniques.” Journal of Magnetic Resonance Imaging 40.3 (2014): 609-615.
[110] C. Ma, Z. Long, D. M. Lanners, D. J. Tradup, C. L. Brunnquell, J. P. Felmlee, D. A. Woodrum, R. E. Watson, N. J. Hangiandreou, and K. R. Gorny. Protocol for testing suitability of compact us imaging systems for use inside MRI suites, and application to one commercial US system. Biomedical Physics & Engineering Express, 2(4):047003, 2016.
[111] Paxinos, G., Watson, C., 2014. The Rat Brain in Stereotaxic Coordinates. Elsevier Academic Press, Amsterdam; Boston.
[112] Shih, Y. Y., et al., 2014a. Dynamic perfusion and diffusion MRI of cortical spreading depolarization in photothrombotic ischemia. Neurobiol Dis, 71, 131-139.
[113] Ajna Borogovac and Iris Asllani, “Arterial Spin Labeling (ASL) fMRI: Advantages, Theoretical Constrains and Experimental Challenges in Neurosciences,” International Journal of Biomedical Imaging, vol. 2012, Article ID 818456, 13 pages, 2012. doi:10.1155/2012/818456
[114] Shih, Y. Y., et al., 2008. Whole-brain functional magnetic resonance imaging mapping of acute nociceptive responses induced by formalin in rats using atlas registration-based event-related analysis. J. Neurosci. Res. 86, 1801-1811.
[115] Lai, H. Y., et al., 2014. Functional MRI reveals frequency-dependent responses during deep brain stimulation at the subthalamic nucleus or internal globus pallidus. Neuroimage 84, 11-18.
[116] Lai, H. Y., et al., 2015. Robust deep brain stimulation functional MRI procedures in rats and mice using an MR-compatible tungsten microwire electrode. Magn. Reson. Med. 73 (3), 1246-51.
[117] Shih, Y. Y., et al., 2013. Ultra high-resolution fMRI and electrophysiology of the rat primary somatosensory cortex. Neuroimage 73, 113-120.
[118] Shih, Y. Y., et al., 2014b. Imaging neurovascular function and functional recovery after stroke in the rat striatum using forepaw stimulation. J. Cereb. Blood Flow Metab. 34 (9), 1483-92
[119] Herscovitch, P., Raichle, M. E., 1985. What is the correct value for the brain—blood partition coefficient for water? J. Cereb. Blood Flow Metab. 5, 65-69.
[120] de Graaf, R. A., et al., 2006. High magnetic field water and metabolite proton T1 and T2 relaxation in rat brain in vivo. Magn. Reson. Med. 56, 386-394.
[121] Shih, Y. Y., et al., 2011. Striatal and cortical BOLD, blood flow, blood volume, oxygen consumption, and glucose consumption changes in noxious forepaw electrical stimulation. J. Cereb. Blood Flow Metab. 31, 832-841.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for generating an ultrasound image of a volume, the method comprising:
detecting a position of each of two or more targets in the volume to be imaged;
generating and directing a single beam of ultrasound energy toward the volume by simultaneously focusing the single beam on each of the two or more the target positions;
detecting the ultrasound energy from each of the two or more the target positions; and
using the detected ultrasound energy to generate an image of the volume to be imaged.

2. The method of claim 1, wherein the each of the two or more targets comprises a contrast agent.

3. The method of claim 2, wherein the contrast agent comprises one or more microbubble, one or more microdroplet, or a combination thereof.

4. The method of claim 1, wherein the detecting a position of each of two or more targets comprises generating and directing a plane wave into the volume.

5. The method of claim 1, wherein detecting a position of each of two or more targets is carried out continually.

6. The method of claim 1, comprising transmitting pulses according to a sequence, optionally a temporal sequence, which simultaneously focuses the single beam on each of the two or more the target positions.

7. The method of claim 1, comprising employing an ultrasound transducer comprising a programmable array configured to transmit pulses according to a sequence, optionally a temporal sequence, that simultaneously focuses the single beam on each of the two or more the target positions.

8. The method of claim 1, comprising repeating the steps at a frame rate of at least about 5,000 frames per second.

9. The method of claim 1, wherein the volume to imaged comprises a tissue in a subject, optionally wherein the tissue is selected from the group consisting of brain, breast and thyroid tissue.

10. The method of claim 1, wherein the volume to be imaged comprises a tumor in a subject, optionally a microvasculature of a tumor, in a subject.

11. An imaging system comprising:
at least one ultrasound transducer configured to detect a position of each of two or more targets in the volume to be imaged, to generate and direct a single beam of ultrasound energy toward the volume by simultaneously focusing the single beam on each of the two or more the target positions, and to detect the ultrasound energy from each of the two or more the target positions; and
a processor programmed to analyze data acquired by the ultrasound transducer from the volume in order to output an image from the volume.

12. The system of claim 11, wherein the at least one ultrasound transducer comprises two or more ultrasound transducers, optionally wherein each ultrasound transducer is configured to detect a position of each of two or more targets in the volume to be imaged, to generate and direct a single beam of ultrasound energy toward the volume by simultaneously focusing the single beam on each of the two or more the target positions, and to detect the ultrasound energy from each of the two or more the target positions.

13. The system of claim 11, comprising a contrast agent, wherein contrast agent is adapted for administration to the volume, such that the each of the two or more targets can comprise a contrast agent.

14. The system of claim 13, wherein the contrast agent comprises one or more microbubble, one or more microdroplet, or a combination thereof.

15. The system of claim 11, wherein the at least one ultrasound transducer is configured to detect a position of each of two or more targets by generating and directing a plane wave into the volume.

16. The system of claim 11, wherein the at least one ultrasound transducer is configured to detect a position of each of two or more targets continually.

17. The system of claim 11, wherein the at least one ultrasound transducer is configured to transmit pulses according to a sequence, optionally a temporal sequence, that simultaneously focuses the single beam on each of the two or more the target positions.

18. The system of claim 11, wherein the at least one ultrasound transducer comprises a programmable array configured to transmit pulses according to a sequence, optionally a temporal sequence, which simultaneously focuses the single beam on each of the two or more the target positions.

19. The system of claim 11, wherein the system is configured to repeat functions at a frame rate of at least about 5,000 frames per second.

20. The system of claim 11, wherein the system is configured to image a volume comprising a tissue in a subject, optionally wherein the tissue is selected from the group consisting of brain, breast and thyroid tissue.

21. The system of claim 11, wherein the system is configured to image a volume comprising a tumor in a subject, optionally a microvasculature of a tumor, in a subject.

22. A non-transitory computer readable medium having stored thereon executable instructions that when executed by the processor of a computer control the computer to perform steps comprising:
detecting a position of each of two or more targets in the volume to be imaged;
generating and directing a single beam of ultrasound energy toward the volume by simultaneously focusing the single beam on each of the two or more the target positions;
detecting the ultrasound energy from each of the two or more the target positions; and
using the detected ultrasound energy to generate an image of the volume to be imaged.

23. A method for generating a blood flow profile in a blood vessel, the method comprising: detecting a position and/or motion of a target in the blood flow in a blood vessel to be imaged in a first ultrasound image and in a second ultrasound image, wherein the first ultrasound image and the second ultrasound image are generated by: (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image; and generating a blood flow profile in the blood vessel based on the position and/or of the target in the first frame and the second frame.

24. The method of claim 23, wherein the target comprises a contrast agent.

25. The method of claim 24, wherein the contrast agent comprises one or microbubble, one or more microdroplet, or a combination thereof.

26. The method of claim 23, wherein the target comprises two or more targets wherein the generating and directing a beam of ultrasound energy toward the blood vessel and focusing the beam on each of the two or more the targets comprises:
generating and directing a single beam of ultrasound energy toward the blood vessel by simultaneously focusing the single beam on each of the two or more the targets.

27. The method of claim 26, comprising transmitting pulses according to a sequence, optionally a temporal sequence, which simultaneously focuses the single beam on each of the two or more the targets.

28. The method of claim 26, comprising employing an ultrasound transducer comprising a programmable array configured to transmit pulses according to a sequence, optionally a temporal sequence, that simultaneously focuses the single beam on each of the two or more the targets.

29. The method of claim 23, comprising repeating the steps (a)-(c) at a frame rate of at least about 5,000 frames per second.

30. The method of claim 23, wherein the blood vessel to imaged comprises a blood vessel in a tissue in a subject, optionally wherein the tissue is selected from the group consisting of brain, breast and thyroid tissue.

31. The method of claim 23, wherein the blood vessel to be imaged comprises a blood vessel of a tumor in a subject.

32. The method of claim 23, 30 or 31, wherein the blood vessel is present in the subject at a depth of at least about 3 centimeters

33. The method of claim 23, wherein generating a blood flow profile comprises determining a velocity of the blood flow, determining a blood vessel pattern, generating a tortuosity measurement for the blood vessel.

34. The method of claim 23, wherein generating a blood flow profile in the blood vessel based on the position and/or of the target in the first frame and the second frame comprises applying a pattern matching function to the ultrasound energy from the target in the first image and the second image.

35. The method of claim 30, wherein the blood vessel is in the brain of a subject, and the method comprises applying a correction that refocuses the ultrasound beam as it propagates through a skull of the subject and/or accounts for one or more variation in skull morphology.

36. The method of claim 35, wherein the correction is applied twice, first when the beam propagates through the skull after being directed toward the blood vessel, and then when the beam propagates from each of the two or more the target positions for detection.

37. The method of claim 23, comprising detecting the position and/or motion of a target in the blood flow in a blood vessel to be imaged in a reference ultrasound image or images and in a subsequent ultrasound image or images, wherein the reference image or images and the subsequent ultrasound image or images are generated by (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image; and generating a blood flow profile in the blood vessel based on the position of the target in the reference image or images and subsequent image or images.

39. An imaging system comprising:
at least one ultrasound transducer configured to detect a position and/or motion of a target in the blood flow in a blood vessel to be imaged in a first ultrasound image and in a second ultrasound image, wherein the first ultrasound image and the second ultrasound image are generated by: (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image; and
a processor programmed to analyze data acquired by the ultrasound transducer to generate a blood flow profile in the blood vessel based on the position and/or of the target in the first frame and the second frame.

40. The system of claim 39, comprising a contrast agent, wherein the contrast agent is adapted for administration to the blood vessel.

41. The system of claim 40, wherein the contrast agent comprises one or microbubble, one or more microdroplet, or a combination thereof.

42. The system of claim 39, wherein the target comprises two or more targets and wherein the at least one ultrasound transducer is configured to generate and direct a single beam of ultrasound energy toward the blood vessel by simultaneously focusing the single beam on each of the two or more the targets.

43. The system of claim 42, wherein the at least one ultrasound transducer is configured to transmit pulses according to a sequence, optionally a temporal sequence, which simultaneously focuses the single beam on each of the two or more the targets.

44. The system of claim 42, wherein the at least one ultrasound transducer comprises a programmable array configured to transmit pulses according to a sequence, optionally a temporal sequence, that simultaneously focuses the single beam on each of the two or more the targets.

45. The system of claim 39, wherein the at least one ultrasound transducer is configured to repeat the steps (a)-(c) at a frame rate of at least about 5,000 frames per second.

46. The system of claim 39, wherein the system is configured to image a blood vessel in a tissue in a subject, optionally wherein the tissue is selected from the group consisting of brain, breast and thyroid tissue.

47. The system of claim 39, wherein the system is configured to image a blood vessel of a tumor in a subject.

48. The system of claim 39, 46 or 47, wherein the blood vessel is present in the subject at a depth of at least about 3 centimeters

49. The system of claim 39, wherein the processor is configured to generate a blood flow profile comprising one or more of a velocity of the blood flow, a blood vessel pattern, and a tortuosity measurement for the blood vessel.

50. The system of claim 39, wherein the processor is configured to apply a pattern matching function to the ultrasound energy from the target in the first image and the second image.

51. The system of claim 46, wherein the blood vessel is in the brain of a subject, and the at least one ultrasound transducer is configured to apply a correction that refocuses the ultrasound beam as it propagates through a skull of the subject and/or accounts for one or more variation in skull morphology.

52. The system of claim 51, wherein the correction is applied twice, first when the beam propagates through the skull after being directed toward the blood vessel, and then when the beam propagates from each of the two or more the target positions for detection.

53. The system of claim 39, at least one ultrasound transducer configured to detect the position and/or motion of a target in the blood flow in a blood vessel to be imaged in a reference ultrasound image or images and in a subsequent ultrasound image or images, wherein the reference image or images and the subsequent ultrasound image or images are generated by (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image.

54. A non-transitory computer readable medium having stored thereon executable instructions that when executed by the processor of a computer control the computer to perform steps comprising:
detecting a position and/or motion of a target in the blood flow in a blood vessel to be imaged in a first ultrasound image and in a second ultrasound image, wherein the first ultrasound image and the second ultrasound image are generated by: (a) generating and directing a beam of ultrasound energy toward the blood vessel, (b) focusing the beam on the target, and (c) detecting the ultrasound energy from the target, wherein steps (a)-(c) are carried out in a manner that provides a super-resolved image; and generating a blood flow profile in the blood vessel based on the position and/or motion of the target in the first frame and the second frame.
